(19) United States
(12) Patent Application Publication
(10) Pub. No.: US 2016/0103487 A1
(43) Pub. Date: Apr. 14, 2016

(54) BRAIN COMPUTER INTERFACE (BCI) SYSTEM BASED ON GATHERED TEMPORAL AND SPATIAL PATTERNS OF BIOPHYSICAL SIGNALS

(71) Applicants: Glen J. ANDERSON, (US); David P. KUHNS, Salem, OR (US); Peter WYATT, San Francisco, CA (US); INTEL CORPORATION, Santa Clara, CA (US)

(72) Inventors: Richard P. Crawford, Davis, CA (US); Glen J. Anderson, Beaverton, OR (US); David P. Kuhns, Salem, OR (US); Peter Wyatt, San Francisco, CA (US)

(21) Appl. No.: 13/994,593

(22) PCT Filed: Mar. 15, 2013
(86) PCT No.: PCT/US13/32037

§ 371 Date: Jun. 14, 2013

(51) Int. Cl. G06F 3/01; A61B 5/117; A61B 5/00; A61B 5/0484; A61B 5/04

(52) U.S. Cl. CPC G06F 3/015; A61B 5/0484; A61B 5/04012; A61B 5/7246; A61B 5/117; A61B 5/742; A61B 2560/0223; A61B 2560/0475

(57) ABSTRACT

Embodiments for providing brain computer interface (BCI) system based on gathered temporal and spatial patterns of biophysical signals are generally described herein. In some embodiments, stimuli are provided to a user. Temporal and spatial patterns of biophysical signals associated with brain activity of a user are gathered in response to providing the stimuli to the user. The gathered temporal and spatial patterns of biophysical signals associated with brain activity of the user are correlated to identify user brain signatures. A processor controlled function based on the user brain signatures identified through correlating of the gathered temporal and spatial patterns of biophysical signals associated with brain activity is performed.

### BACKGROUND

Communication and control of the environment is important to everyday life. In particular, disabled people have gone to extraordinary lengths to communicate. A Brain Computer Interface (BCI) enables direct communication between the brain and a computer or electronic device. BCI could also be applied in applications where existing communication methods exhibit shortcomings, e.g. in noisy industrial applications, military environments where stealth and movement are constrained, etc. In the consumer market. BCI may provide advantages as a gaming or entertainment interface or may speed existing or enable entirely new computer-user interactions.

Regardless of function, each part of the brain is made up of nerve cells called neurons. As a whole, the brain is a dense network that includes about 100 billion neurons. Each of these neurons communicates with thousands of others in order to regulate physical processes and to produce thought. Neurons communicate either by sending electrical signals to other neurons through physical connections or by exchanging chemicals called neurotransmitters. When they communicate, neurons consume oxygen and glucose that is replenished through increased blood flow to the active regions of the brain.

Advances in brain monitoring technologies allow observations of the electric, chemical, fluidic, magnetic, etc. changes as the brain processes information or responds to various stimuli. Research continues in brain computer interface (BCI) systems that could provide new communication and control options for a wide variety of users and applications. Through brain activity monitoring, a database of characteristic mental profiles may be gathered.

Device and data security threats are ubiquitous and a very high value has been assigned to highly accurate and precise authentication systems and methods. While several forms of biologically distinct signatures or biometrics are employed in authentication systems (fingerprints, retinal patterns, voice characteristics, etc.), there is very little exploitation involving the uniqueness in brains as an authentication technique.

Some BCI systems rely on electroencephalography (EEG), which is characterized by relatively high temporal resolution, but also by relatively low spatial resolution. Further research in BCI systems is underway addressing the many questions and challenges involving their reliable use.

### BRIEF DESCRIPTION OF THE DRAWINGS

FIG. 1 illustrates a system for providing psychological and sociological matching through correlation of brain activity similarities according to an embodiment;

FIG. 2 illustrates a system that compares the brain anatomy and activity to known information and prerecorded signatures to identify and assign a probability of uniqueness to an individual, according to an embodiment;

FIG. 3 illustrates a consumer grade wearable system for gathering BCI input, according to an embodiment;

FIGS. 4a-c illustrate components of a neuroimaging device, according to an embodiment;

FIG. 5 is a flowchart of a method for providing control of computing experiences according to an embodiment:

FIGS. 6a-b illustrate a visual search according to an embodiment;

FIG. 7 illustrates a BCI system for providing telepathic search according to an embodiment;

FIG. 8 illustrates wireless telepathic communication using a BCI system according to an embodiment;

FIG. 9 is a networked system for performing telepathic contextual search according to an embodiment;

FIG. 10 is a flowchart for providing telepathic augmented reality using a BCI system according to an embodiment;

FIGS. 11 a-d show an example of AR presentation and control according to an embodiment;

FIG. 12 illustrates a system that may provide telepathic augmented reality according to an embodiment;

FIG. 13 illustrates a cortical representation of visual space used to represent mental desktop space provided by a BCI system according to an embodiment;

FIG. 14 is a diagram of the optic system according to an embodiment;

FIG. 15 shows the physiologically segregated sections of visual space according to an embodiment;

FIG. 16 is a model of a human cortex showing the primary motor cortex and the primary sensory cortex as utilized by a BCI system according to an embodiment;

FIG. 17 is a model demonstrating the topographical organization of the primary motor cortex on the pre-central gyrus of the human cortex;

FIG. 18 illustrates a user interface for assigning BCI measures and other modalities to applications according to an embodiment;

FIG. 19 shows a BCI system that accepts BCI inputs and other modality inputs according to an embodiment:

FIG. 20 illustrates a flowchart of a method for determining user intent according to an embodiment;

FIG. 21 is a flowchart of a method for assigning BCI input for controlling an application according to an embodiment;

FIG. 22 is a flowchart of a method for adjusting contextual factors by the BCI system according to an embodiment; and

FIG. 23 illustrates a block diagram of an example machine for providing a brain computer interface (BCI) system based on gathered temporal and spatial patterns of biophysical signals.

### DETAILED DESCRIPTION

According to embodiments described herein, brain/skull anatomical characteristics, such as gyrification, cortical thickness, scalp thickness, etc., may be used for identification/authentication purposes. Measured stimuli/response brain characteristics, e.g., anatomic and physiologic, may be translated into specific patterns to categorize a brain for identification and/or authentication purposes. People may be correlated according to similarities in brain activity in response to stimuli. Information on other brain signatures, e.g., anatomic and physiologic, and comparisons to similar brains may be used to predict a brain response to a new stimulus and for identification and/or authentication purposes. Brain identification and/or authentication techniques in combination with other identification and/or authentication techniques, e.g., password, other biometric parameters, may be used to increase the identity/authentication sensitivity and specificity.

More recent technological advances in sensing brain or neuronal activity signal present opportunities for the creation of more sophisticated BCI usages and systems. Based on gathered temporal and spatial patterns of biophysical signals, it is now possible to measure and identify psychological states or mental representations of a person. For example, temporal and spatial patterns of biophysical signals may be obtained through, but not limited to, electrical, fluidic, chemical, magnetic sensors.

Examples of devices that gather electrical signals include electroencephalography (EEG). EEG uses electrodes placed directly on the scalp to measure the weak (5-100 μV) electrical potentials generated by activity in the brain. Devices that measure and sense fluidic signals include Doppler ultrasound and devices that measure chemical signals include functional near-infrared spectroscopy (fNIRS). Doppler ultrasound measures cerebral blood flow velocity (CBFV) in the network of arteries that supply the brain. Cognitive activation produces increases in CBFV within these arteries that may be detected using Doppler ultrasound. fNIRS technology works by projecting near infrared light into the brain from the surface of the scalp and measuring optical changes at various wavelengths as the light is refracted and reflected back to the surface. The fNIRS effectively measures cerebral hemodynamics and detects localized blood volume and oxygenation changes. Since changes in tissue oxygenation associated with brain activity modulate the absorption and scattering of the near infrared light photons to varying amounts, fNIRS may be used to build functional maps of brain activity. Devices that measure magnetic signals include magnetoencephalography (MEG). A MEG measures magnetic fields generated by the electrical activity of the brain. MEG enables much deeper imaging and is much more sensitive than EEG because the skull is substantially transparent to magnetic waves.

Through the use of biophysical sensor devices, as described above and others, temporal and spatial patterns of biophysical signals may be used to measure and identify psychological states or mental representations of a person to reveal information, such as cognitive workload, attention/distraction, mood, sociological dynamics, memories, and others. Utilization of these data unlocks a new frontier for opportunities in human-machine interaction.

Businesses, political institutions, and society place a high value on finding like-minded people for purposes that include marketing, messaging, and social networking. Systems and techniques that may assist in the identification of like-minded (or conversely, unlike-minded) people would accordingly be highly valued. Information on regarding how brains respond to similar stimuli may be used to ascertain and score the “mental similarity” between different people or groups of people. This may be used in conjunction with other measures of mental, personality, or sociological traits to create more sophistication to the matching assessment.

By instrumenting people with brain monitoring devices and then taking them through a series of imagined or thought-provoking experiences (through any imagined sensory channel, visual, auditory, tactile, etc.), the resulting spatial and temporal brain activity patterns may be captured and characterized. The degree to which the brain activity of different people responds similarly would provide a measure of mental similarity. Conversely, the degree to which the brain activity of people responds dissimilarly to the same stimuli would provide measures of mental dissimilarity. The collection of responses to the set of stimuli may be used to build characteristic mental profiles and serve to establish models of mental predilections that would be equivalent to concepts, such as the Meyers-Briggs® or Five Factor Model (FFM), for characterizing personality traits.

The political, mental, or social compatibility (or incompatibility) or people may be predicted using temporal and spatial patterns of biophysical signals and stimuli response data based on the theory that certain similarities or correlations of mental responses make for better pairings. This comparison to others could happen through a web-based system and infrastructure to be used as part of dating services, e.g., Match.com®.

Specific examples where this idea may prove useful include a comparison of mental profiles with job satisfaction information to help identify and predict potential career matches (or mismatches), with relationship satisfaction to help identify and predict potential social compatibility (or incompatibility), with political inclinations to help identify and predict political party alignment (or misalignment), with product usage, satisfaction, or interest to help identify marketing targets, etc.

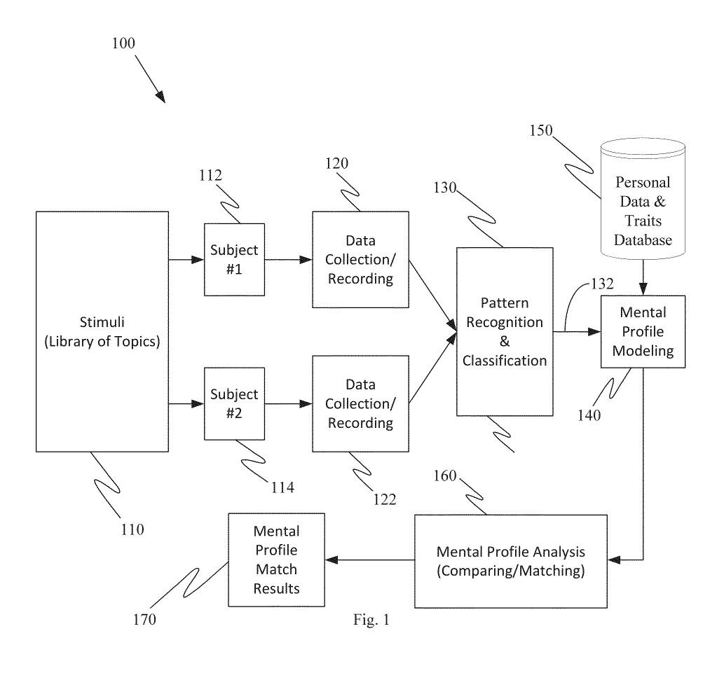

FIG. 1 illustrates a system 100 for providing psychological and sociological matching through correlation of brain activity similarities according to an embodiment. The system collects spatial and hemispherical information to define inputs to a BCI system. A library of stimuli 110 is provided to elicit brain activity in subjects 112, 114. The library of stimuli 110 include sets of stimuli involving any of various media designed to engage the brain, e.g., pictures, films, audio tracks, written or spoken questions or problems. The stimuli options are numerous and a library of specific stimuli 110 foci is imagined that could range from the general, e.g., designed to test emotional sensitivity, to the very specific, e.g., orientations on family and child-rearing. The brain activity is recorded by a data collection and recording system 120, 122 as the stimuli are presented to the subjects 112, 114. Neuroimaging devices that measure the resulting brain activation patterns may include EEG, fNIRS, MEG, MRI (magnetic resonance imaging), ultrasound, etc. However, embodiments are not meant to be limited to measurement systems specifically mentioned herein.

The recorded brain activity is then processed using a pattern recognition and classification system 130. A pattern recognition system 130 processes the recorded brain activity to characterize and classify the brain activation pattern. There are numerous techniques and algorithms from the field of pattern recognition that may be applied including classification, clustering, regression, categorical sequence labeling, real-valued sequence labeling, parsing, Bayesian networks, Markov random fields, ensemble learning, etc. Further methods for obtaining or analyzing brain activity: modified Beer-Lambert Law, event-related components, multi-voxel pattern analysis, spectral analysis, the use of MVPA (multi-voxel pattern analysis) on fNIRS, etc. Simple brain EEG signal characterization may be used for identification purposes. General purpose pattern recognition techniques and algorithms may be implemented. The pattern and classification results 132 are combined, at a mental profile modeling system 140, with personal data and other traits from a database 150 to develop mental profile models of the subjects 112, 114. The mental profile modeling system 140 thus creates a model that combines the brain pattern recognition results with other personal data and traits, such as gender, age, geographic location, genetic information, etc., to build a mental profile as a function of the specific stimuli.

The personal data and other traits from database 150 may be obtained through questionnaires, observation, etc. and maintained in a personality trait database. The mental profile modeling system 140 produces a mental profile match of a subject by comparing the mental profile of a subject with a database of other mental profiles. A mental profile analysis system 160 correlates probabilities between subjects. The mental profile analysis system 160 calculates the statistics and probability of mental match for any of a range of topics, e.g., social, problem solving, music genre affinity, financial orientation, etc. General purpose statistical techniques may be used to translate pattern recognition into probabilistic relationships given known conditions.

Accordingly, the system 100 translates recorded brain activity patterns in response to stimulus 110 into a characteristic mental profile for that stimulus. A library of stimuli 110 is translated into a library of mental profiles for each individual. The mental profiles also include the integration of personal data and traits from database 150. The mental profile analysis system 160 derives the similarity or dissimilarity of mental profiles based on the degree of similarity or dissimilarity of pattern matching results between two people for a stimulus or set of stimuli. This result, a mental profile match result 170 represents a probabilistic score of a “mental match.”

BCI systems may also be used to provide user identification and authentication using brain signatures. Everyone has unique brain anatomical model and physiological characteristics that are a function of their genetic, environmental, and situational influences that can be leveraged for identification and authentication purposes.

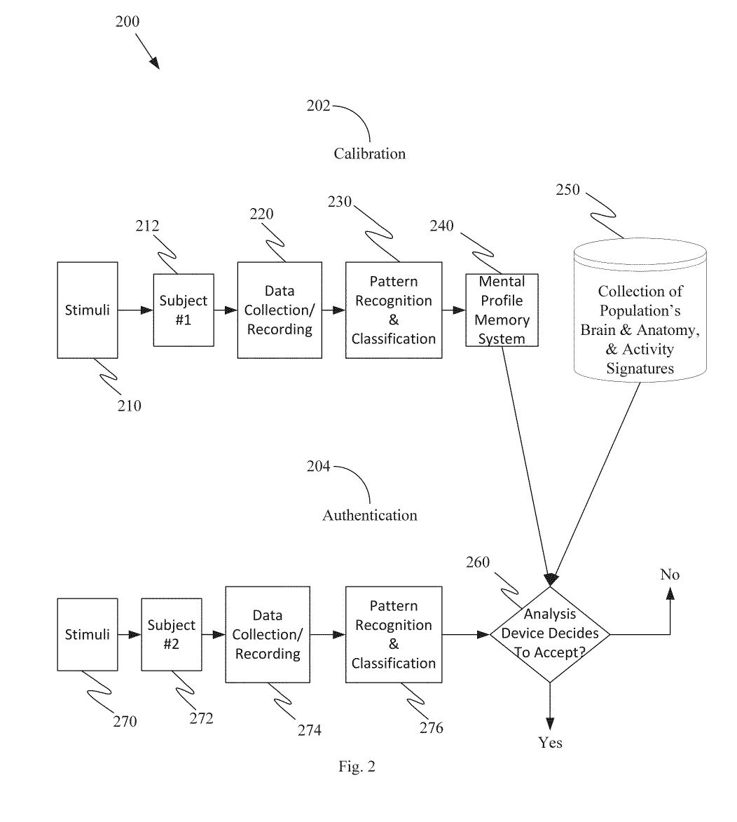

FIG. 2 illustrates a system 200 that compares the brain anatomy and activity to known information and prerecorded signatures to identify and assign a probability of uniqueness to an individual, according to an embodiment. Accordingly, the brain anatomical model and physiological uniqueness may be used for security and/or authentication purposes. In FIG. 2, a calibration process 202 and an authentication process 204 based on brain responses to activity are shown. In the calibration process 202, stimuli 210 are provided to a first subject 212. The stimuli 210 may include images, statements, or questions that are presented to a user that would incite certain characteristic brain activity responses. The response of the subject is measure by data gathering and recording system 220. The data gathering and recording system 220 also measure activity associated with brain anatomy and activity. Neuroimaging devices that measure the resulting brain activation patterns may include EEG, fNIRS, MEG, MRI, ultrasound, etc.

The data associated with brain anatomy and activity is provided to a pattern recognition device 230 that analyzes the associated with brain anatomy and activity to identify patterns. Anatomic characteristics of the brain. e.g., gyrification, cortical thickness, etc., are identified. Again, there are numerous techniques and algorithms from the field of pattern recognition that may be applied including classification, clustering, regression, categorical sequence labeling, real-valued sequence labeling, parsing, Bayesian networks, Markov random fields, ensemble learning, etc. Further methods for obtaining or analyzing brain activity: modified Beer-Lambert Law, event-related components, multi-voxel pattern analysis, spectral analysis, the use of MVPA on fNIRS, etc. Simple brain EEG signal characterization may be used for identification purposes. General purpose pattern recognition techniques and algorithms may be implemented.

The brain measurements are stored in a memory profile memory system 240. A database 250 maintains a collection of a population's brain anatomy and activity signatures. During the authentication process 204, stimuli 270 are provided to a second subject 272. The second subject 272 may be the first subject 212 or another subject having data maintained in the database 250. The response of the subject is measured by data collection and recording system 274. The response may include data associated with brain anatomy and activity. Again, brain anatomy may be obtained using technology such as ultrasound, and/or EEG, fNIRS, MEG, MRI, etc. The data associated with brain anatomy and activity is provided to a pattern recognition device 276 that analyzes the associated with brain anatomy and activity to identify patterns. Again, anatomic characteristics of the brain, e.g., gyrification, cortical thickness, etc., are identified.

An analysis device 260 receives the results from the pattern recognition device, the previously processed brain measurements and prediction data associated with the subject from the database that maintains a collection of a population's brain anatomy and activity signatures. The analysis device 260 determines whether the subject being authenticated correlates to the subject's previously processed brain measurements and prediction data. The brain anatomy and activity patterns are thus compared to known or predicted signatures collected during calibration sessions, previous signatures collections, or predicted a priori from a library of ‘similar’ brain signatures. The analysis device 260 assigns a confidence of authenticity to authentication. The confidence of authenticity to the authentication may be based on statistical techniques to translate pattern recognition into probabilistic relationships given known conditions.

If the analysis device 260 determines that the response from the subject 272 is not acceptable, the subject may be rejected. However, if the brain measurements of the subject 272 being authenticated correlates with the subject's previously processed brain measurements and prediction data, the subject may be accepted. These brain signature identification techniques may be used in combination with other indeterminate authentication methods, e.g., handwriting recognition, to improve the sensitivity and specificity of the authentication method.

Accordingly, the system may be used to compare the brain anatomy and activity to known information and prerecorded signatures to identify and assign a probability of uniqueness. Alternatively, the system may use a potentially more discriminating and secure approach that may involve a series of memorized thoughts (e.g., child, car, waterfall), patterns of muscle activation (e.g., jump, serve a tennis ball, play a song on a piano), or imagined activities (e.g., pet a cat, solve the equation 13×14, eat banana) that a user would run through mentally to incite certain characteristic brain activities.

In another embodiment, a BCI system may be used to allow users to control a processing device, such as a computer, laptop, mobile phone, tablet computer, smart television, remote controls, microwaves, etc. A BCI system may be used to direct devices to carry out activities by associating mental conditions with media and search services. Current BCI systems rely on EEG, which is characterized by relatively fine temporal resolution but also relatively low spatial resolution. The low spatial resolution limits is not compatible with certain analysis techniques that have been shown to be useful for extracting high-level information. While a product created today based on existing technologies may not be good enough for precision applications, the technology is already available to provide entertainment-level implementations.

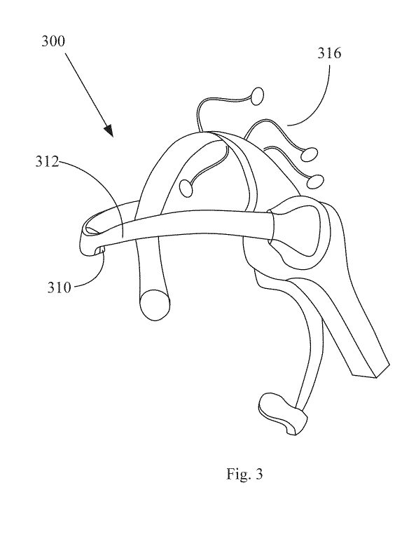

FIG. 3 illustrates an example of a wearable system 300 for gathering BCI input according to an embodiment. A wearable brain system 300 may utilize an EEG sensor 310 that is held in place against the forehead with a headband 312. Alternatively or in addition to EEG sensor 310, the wearable system 300 may include several electrodes 316 that may be attached to the user's head to detect EEG signals. The wearable brain imaging device 300 may allow a user to grossly control one factor, e.g., the level of air pressure that a small fan outputs, by measuring electrical activity in the brain. The nature of EEG limits the spatial resolution to gross statistical estimates of scalp distributions that typically lead BCI-EEG devices to utilize spectral analysis to analyze and tease apart the unique frequency bands contained within the EEG signal.

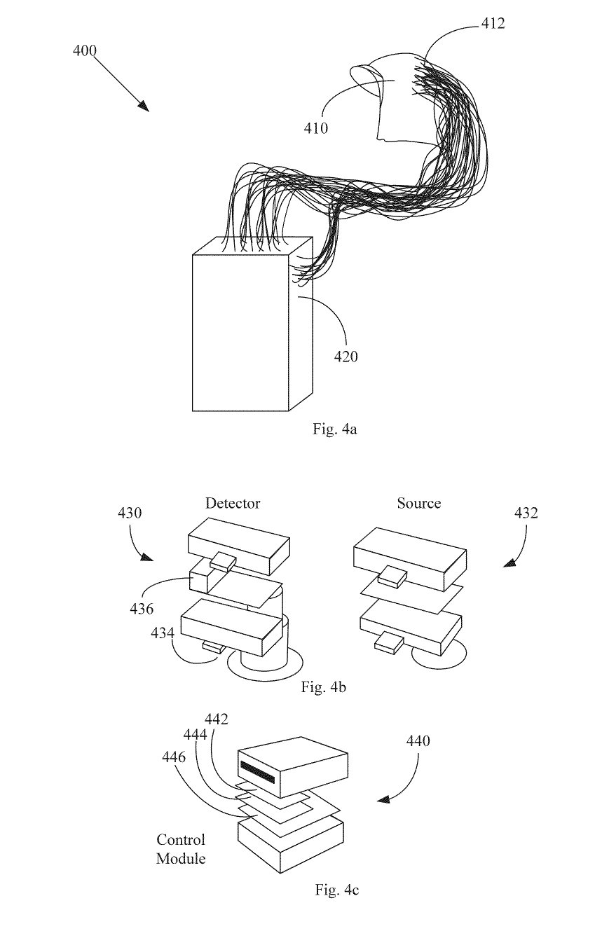

FIGS. 4a-c illustrate components of a neuroimaging device 400, according to an embodiment. Far more sophisticated BCI input sensors move away from EEG to neuroimaging approaches that provide higher spatial resolution that is similar to MRI or PET scans. Optical brain imaging or even a combination of optical and EEG provide a system with the spatial and temporal resolution used for distinguishing hundreds or even thousands of unique activation patterns. In FIG. 4a, a hat or helmet 410 is provided for the subject that includes a plurality of sensors and sources 412. The sensors and sources 412 are provided to a processing device 420 that may be coupled to the subject. FIG. 4b shows a detector 430 and a source 432 that is used in the neuroimaging device 400. The sensors 432 may include EEG. A near-infrared spectroscopy (NIRS) detector 430 may also be used. FIG. 4c illustrates the control module 440.

One embodiment for using a BCI system to provide control computing experiences involves telepathic search. By monitoring BCI patterns while a user is exposed to various media, e.g., music or images, the BCI system may create a database of associations. Subsequently, when the user is in a search mode, mental imagery may recreate those brain activity patterns to help make the search more efficient. Another embodiment providing control computing experiences involves telepathic communication. By training the two or more users on the same set of media, while monitoring brain activity patterns, the system could create a common mental vocabulary that users could use to communicate with each other. Another embodiment providing control computing experiences involves telepathic augmented reality. Users may train mental imagery that is paired with 3D models and/or animation of those models to perform specific actions. Thus, by thinking about the model, the user may cause the 3D model or animation to appear while viewing through a device with AR capability.

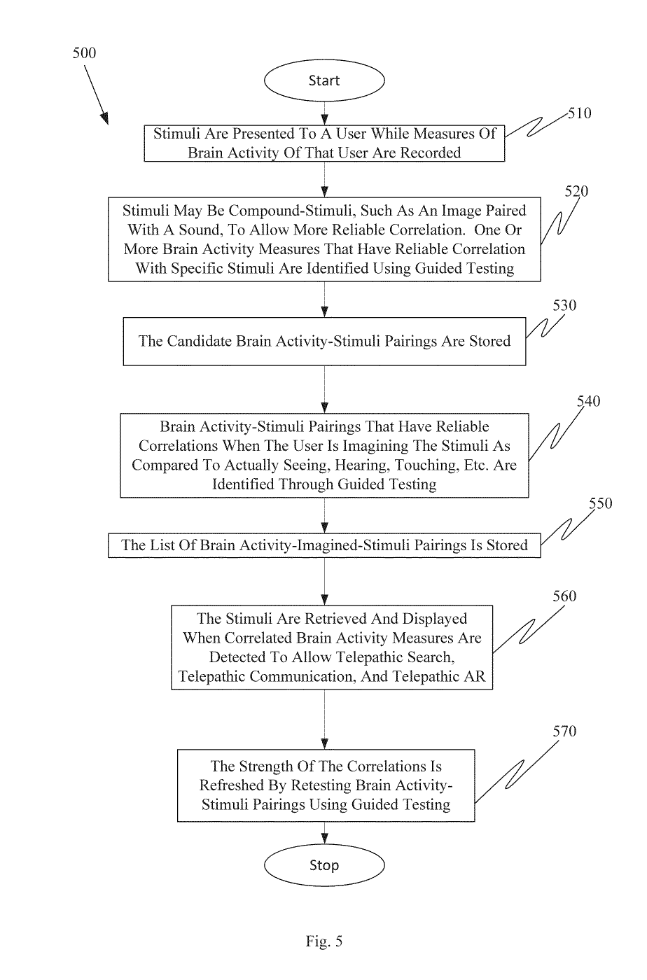

FIG. 5 is a flowchart of a method 500 for providing control of computing experiences according to an embodiment. Stimuli are presented to a user while measures of brain activity of that user are recorded 510. Stimuli may be compound-stimuli, such as an image paired with a sound, to allow more reliable correlation. One or more brain activity measures that have reliable correlation with specific stimuli are identified using guided testing 520. The brain activity measures may be one or more types, e.g., fNIRS and EEG. The candidate brain activity-stimuli pairings are stored 530. Brain activity-stimuli pairings that have reliable correlations when the user is imagining the stimuli as compared to actually seeing, hearing, touching, etc. are identified through guided testing 540. The list of brain activity-imagined-stimuli pairings is stored 550. The stimuli are retrieved and displayed when correlated brain activity measures are detected to allow telepathic search, telepathic communication, and telepathic AR 560. The strength of the correlations is refreshed by retesting brain activity-stimuli pairings using guided testing 570.

With regard to a telepathic search, users typically know what content they are searching for and provide input search terms into a search tool. For example, to search for the song “Song 1” in a library, the user provides input search terms that overlap with song title, artist, album title, genre or others. However, users may have varying levels of search literacy for complex searches or may have fuzzy concepts of searches requests that too poorly define an effective search. This consequently produces poor search results. However, a telepathic search performed according to an embodiment allows users to perform a hands-free search against an image or music database by using a user's mental visualization. A telepathic search according to an embodiment allows for searches such as image searches, video searches, music searches, or web searches. A telepathic search may also allow a user to perform a search without knowing the actual word.

The BCI system builds on the concept of matching the unique patterns of thought to a database of content that is categorized to a user's brain patterns that emerge in response to basic elements of a thought, e.g., movement, light/dark patterns, attentional settings, etc. Once the user's brain patterns are recorded and correlated, the BCI system reconstructs thoughts from the brain patterns alone. When the users initiate a search, the new thought would be matched to known elements from previous thoughts and content stored in the database. Search results may be weighted based on the number of elements in the new thought that match with elements known to be associated with content in the database. The search results would be seemingly telepathic in the way that a user could think a thought and have the BCI system perform a telepathic search that return results matching the thought.

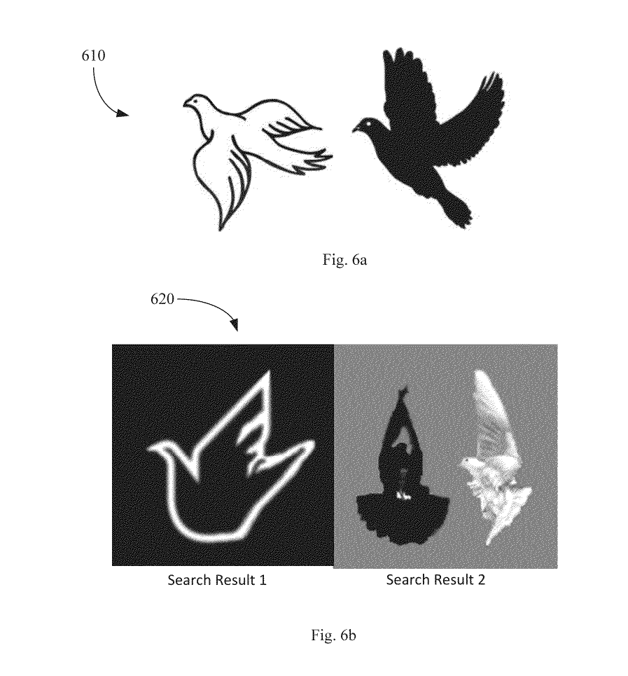

One example may include a user that is searching for an image. The memory is stored as a mental representation of the image, which may or may not be easily translated into words. Perhaps the image is a picture of a white dove followed by a black dove 610 as represented in FIG. 6a.

Internet searches for the aforementioned picture may verbally be searched for with, “white dove followed black dove.” Translating a purely visual concept into a text search yields spurious results 620 as shown in FIG. 6b. Translating a visualization into a visual search is not only intuitive, but holds the potential for more effective searches where verbal information does not easily translate from the output into a text-based search.

Another example where a telepathic search would yield superior results for a non-verbal search than a text-based search involves a search for music. For example, a user may want Animation's 1984 masterpiece “Obsession,” but cannot remember the artist, song title, album or lyrics. The user could think of the sounds of the song and a BCI system performing a telepathic search provides results of music that matches brain activations to the user's thoughts of the sound of “Obsession” without the user providing text inputs. A BCI system may perform such a search by matching those patterns of brain activity from the learning phase with brain activations produced by thinking of the song.

Cognitive psychology provide strong support for the neural network model, which proposes that representations in the brain are stored as patterns of distributed brain activity co-occurring in a particular temporal and spatial relationship. For example, a response to a particular input, such as a picture, results in a distribution of neuronal activity that is distributed across the brain in a specific pattern in time and cortical spatial location in your brain, which produces as an output the visual representation of the input.

Along these same lines, the psychophysical process of stimulus perception begins in the brain with the individual components signaled in the brain, then reassembled based on the elements that fall within attention. For example, when a viewer perceives an object, the color information, shape information, movement information, etc. initially enters the brain as individual components and attention or another mechanism binds the elements together to form a coherent percept. These concepts are important because a stimulus is not represented as a whole object or in a single, unified portion of the brain.

A telepathic search according to an embodiment may be implemented using techniques like multi-voxel pattern analysis (MVPA). MVPA builds on the knowledge that stimuli are represented in a distributed manner and perceived as a reconstruction of their individual elements. MVPA is a quantitative neuroimaging methodology that identifies patterns of distributed brain activity that are correlated with a particular thought such as perceiving a visual stimulus, perceiving an auditory stimulus, remembering three items simultaneously, attending to one dimension of an object while not focusing on another, etc. MVPA identifies the spatial and temporal patterns of activity distributed throughout the brain that identify complex mental representations or states. The mental representations may be cognitive activities such as memory activities, such as retrieving a long-term memory or representations of perceptual inputs including auditory stimulus. MVPA traditionally utilizes the temporal correlations between brain activity measured in volumetric pixels, i.e., voxels, that become active at a given moment in response to a stimulus or as part of a narrowly defined cognitive activity, e.g., long-term memory retrieval. Temporal and spatial patterns of biophysical signals may also be used to measure and identify psychological states or mental representations of a person to reveal information, such as cognitive workload, attention/distraction, mood, sociological dynamics, memories, and others.

MVPA may identify a person's unique patterns of activations in response to particular stimulus, then reconstruct that stimulus the patterns of brain activation alone. For example, video from a users' brain activations have may be reconstructed after the MVPA had been trained to learn the brain responses from the video. First, users may be shown video clips, and then each user's idiosyncratic pattern of activity in response to each video may be analyzed using MVPA to identify brain activity associated with elements of the video. Following the learning episode, the brain activity alone may identify enough elements from the video to reconstruct it by matching the brain activity to elements of videos stored in a database.

However, MVPA is mainly applied to MRI neuroimaging. MRI is a powerful neuroimaging technique, but it relies on large super-conducting magnets that make it an impractical brain imaging device in mobile settings. Optical imaging techniques such as fNIRS are relatively nascent but provide the potential for low-cost, wearable solutions that may be extensible to a wide variety of usages and applications.

According to an embodiment, MVPA and fNIRS may be combined to offer a viable analysis approach in MVPA with a viable wearable device to provide novel BCI-software interactions and functionality that is able to distinguish among dozens to potentially hundreds of brain activity patterns.

For a BCI system providing telepathic search, a learning phase is used to learn brain activation patterns in response to stimuli and a search phase is used to match mental representations to searchable content. In the learning phase, the system identifies stable patterns of brain activity in response to a given type of content, e.g., video, music, etc. The patterns are categorized in ways relevant for the type of content, e.g., image properties for pictures or video. Again, neuroimaging devices that may be used include INIRS, EEG, MEG, MRI, etc. Methodologies for obtaining or analyzing brain activity may again include modified Beer-Lambert Law, event-related components, multi-voxel pattern analysis, spectral analysis, use of MVPA on fNIRS.

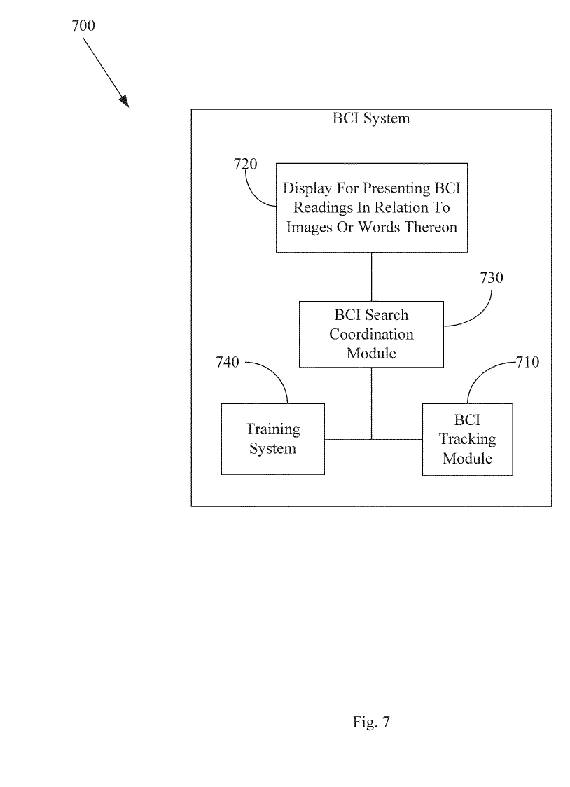

FIG. 7 illustrates a BCI system 700 for providing telepathic search according to an embodiment. In FIG. 7, a BCI Tracking module 710 monitors brain activity readings in relation to images or words on a display 720. A BCI search coordination module 730 retrieves earlier BCI-word associations while the user is engaged with performing a search. Those associations are used to weight or order the search results. A training system 740 displays stimuli, e.g., sight, sound, smell, tactile, etc., or a combination, while measuring a user's brain activity, thus allowing BCI measures to be associated with particular images or words.

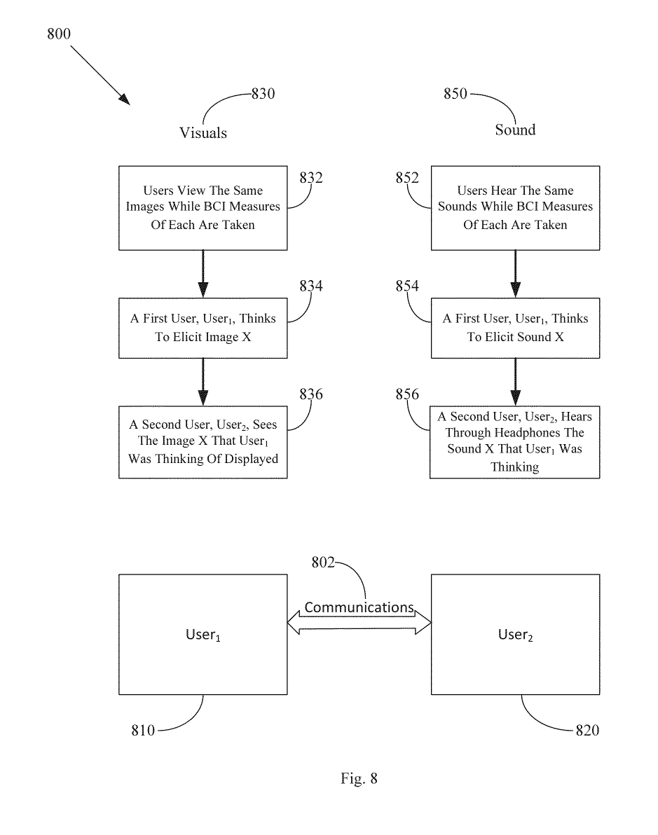

FIG. 8 illustrates wireless telepathic communication using a BCI system 800 according to an embodiment. In FIG. 8, wireless communication 802 is provided between two subjects, first user, user₁ 810 and second user, user₂ 820. The BCI system according to an embodiment enables users to communicate words and symbols using brainwaves and other brain activity measures. Users first have their BCI systems trained on a common set of brain activity measures in reaction to the same stimuli, e.g., as explained above with regard to telepathic searching. Then, when one user uses thought patters related to that stimuli, the BCI system alert the other user by displaying that same stimuli, thus allowing a sort of“mind reading.” The user interface may appear like a text chat UI with simple words and symbols. The user interface may be an audio-based or tactile-based system.

The wireless communication may include visual 830 and/or sound 850. For visuals, users view the same images while brain activity measures of each are taken 832. A first user, user₁ 810, thinks to elicit image X 834. A second user, user₂ 812, sees the image X that user₁ was thinking of displayed 836.

For sound, users hear the same sounds while brain activity measures of each are taken 852. A first user, user₁ 810, thinks to elicit sound X 854. A second user, user₂ 812, hears through headphones the sound X that user1 810 was thinking 856. The sending user may be identified on a UI. The user could think to choose a recipient of the message.

Again, neuroimaging devices that may be used include fNRS, EEG, MEG, MRI, etc. Methodologies for obtaining or analyzing brain activity may again include modified Beer-Lambert Law, event-related components, multi-voxel pattern analysis, spectral analysis, etc.

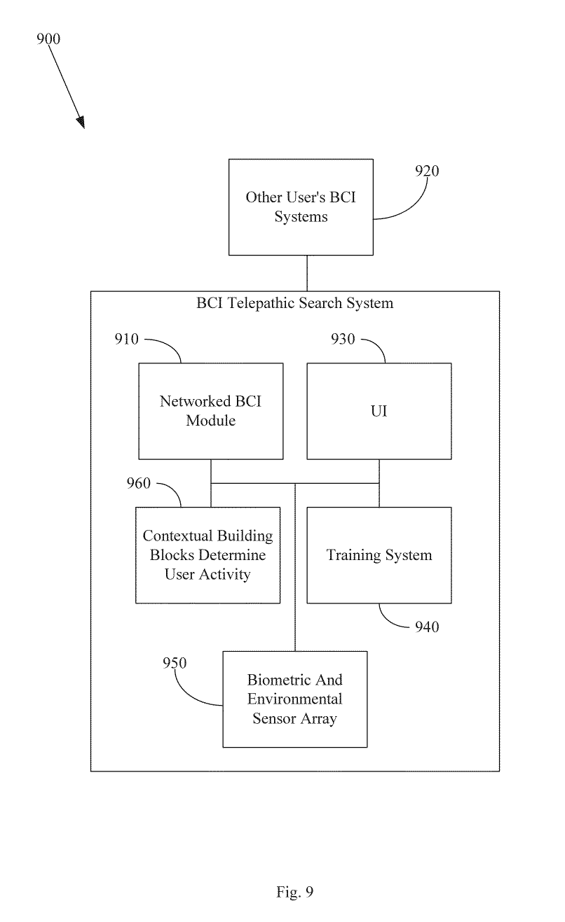

FIG. 9 is a networked system 900 for performing telepathic contextual search according to an embodiment. In FIG. 9, a networked BCI module 910 monitors wireless transmissions from other user's BCI systems 920. A UI 930, such as a chat interface, displays stimuli associated with brain activity measures from another person. The stimuli may also include other senses or combinations, such as sound, words, tactile, etc. A training system 940 displays stimuli, such as sight, sound, smell, tactile, etc., or a combination, to a user the user's brain activity is measured. This allows brain activity measures to be associated with particular images. A biometric and environmental sensor array 950 may be used to provide stimuli and obtain brain activity measurements. Contextual building blocks 960 may be developed that determine user activity, such as walking, running, and talking with someone. The sensor array 950 may be mounted on the user's head.

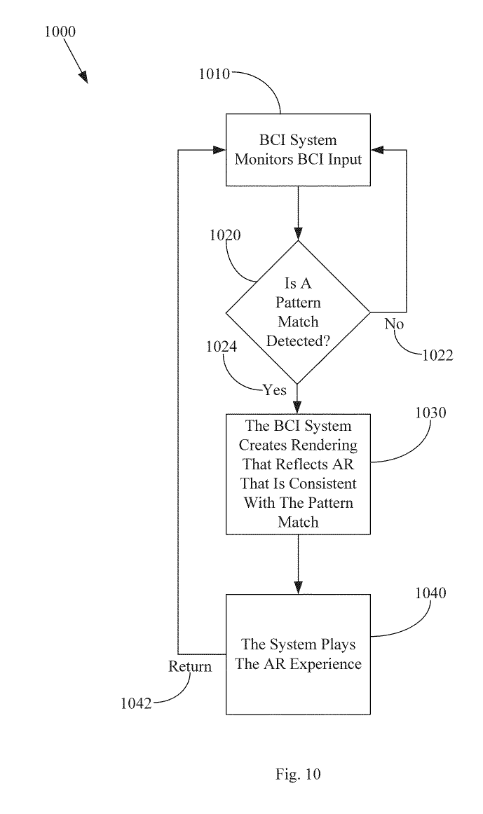

FIG. 10 is a flowchart 1000 for providing telepathic augmented reality using a BCI system according to an embodiment. The BCI system enables users to see, hear and feel augmented reality (AR) objects by consciously directing thought. AR objects may be presented by monitoring BCI inputs and presenting corresponding AR experiences that the user may not purposefully invoke. As explained in previous related disclosures, a BCI system is trained to associate brain activity measures with certain images, thus allowing the image to later be displayed if the user creates a matching brain activity pattern.

In FIG. 10, a BCI system monitors BCI input 1010. A determination is made whether a pattern match is detected 1020. If not, then at 1022 the system continues to monitor BCI input and analyze the BCI input for matches. If a pattern match is detected, then at 1024 the BCI system creates rendering that reflects AR that correlates with the pattern match 1030. The system then plays the AR experience 1040. Thereafter, the BCI process may return 1042 to continue to monitor BCI input and analyze the BCI input for matches.

The AR experience is launched as a result of the monitoring by the BCI system. The AR experience may be visual, audio, tactile, or any other sense-based experience. Further, the users may direct the movement of AR characters through thought. This allows users to play a game in which they control AR characters that move or race. Moreover, the BCI system may monitor the environment for cues that could interact with current BCI input. For example, the system could perform object recognition, and if the user produces a brain activity measure that relates to cartoons, a cartoon version of the identified object may be presented. In another embodiment, the user may invoke the 3D orienting of an object by thinking about its position. Simple systems, e.g., MINDBENDER® allow a user to move objects through the use of concentration. However, these simple systems do not involve AR presentation or control.

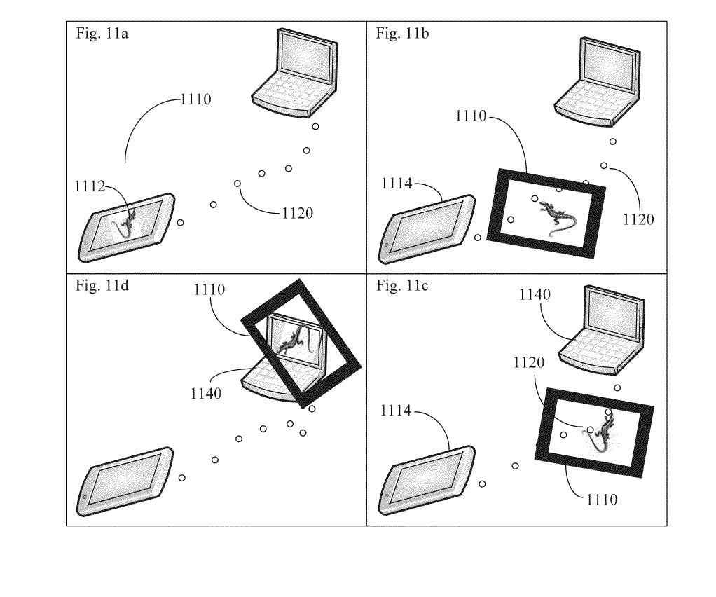

FIGS. 11 a-d show an example of AR presentation and control according to an embodiment. The AR 1110 in FIG. 11a may be presented when the user produced a brain activity measure that corresponded to a lizard 1112. In this example, the user lays out a trail of markers 1120, produces the brain activity match with the lizard 1112, and then watches as the AR experience is displayed. In FIG. 11b, the AR 1110 has moved from the tablet 1114 onto the trail of markers 1120. In FIG. 11c, the AR 1110 moves even farther along the trail of markers 1120 towards the laptop 1140. In FIG. 11d, the AR 1110 has reached the laptop 1140. The display in FIGS. 11 a-d may be through a phone, a head-mounted display or another sensory modality.

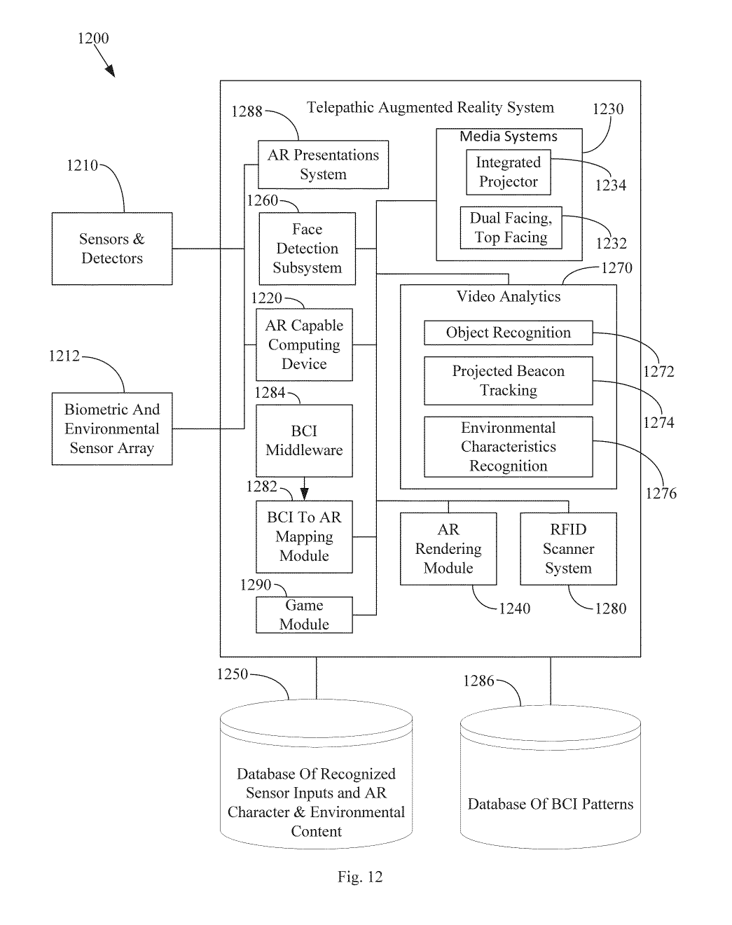

FIG. 12 illustrates a system 1200 that may provide telepathic augmented reality according to an embodiment. Yet again sensors and detectors 1210, such as fNIRS, EEG, MEG, modified Beer-Lambert Law, event-related components, etc. may be utilized along with a biometric and environmental sensor array 1212. The brain activity sensor array may be mounted on the user's head. In an embodiment, an AR capable computing device 1220 may be used with media systems 1230 that may include dual facing cameras 1232, of which one may be a top facing camera.

An AR rendering module 1240 may automatically make AR characters blend with the environment in convincing ways. A database 1250 of recognized sensor inputs is used and AR character and AR environmental content are implemented. A face detection subsystem 1260 may be provided to identify the face of a subject. Further, video analytics 1270 may include object recognition 1272, projected beacon tracking 1274 and environmental characteristics recognition 1276, e.g., recognizing horizontal surfaces to bound AR character actions so it does not goes through the floor. An RFID scanner system 1280 may be used for scanning objects with embedded tags. An integrated projector 1234 may also be utilized.

The BCI system 1200 may further include a BCI to AR mapping module 1282 that receives input from BCI middleware 1284 and maps it to an AR experience. A database 1286 of brain activity patterns provide matches to AR experiences. These matches may be general for users “out of the box,” or they may be created through a matching process. An AR presentations system 1288 may be connected to BCI sensors 1210, and may be wireless or wired. Also, a game module 1290 that allows user to compete in “mind control” of AR characters may be implemented.

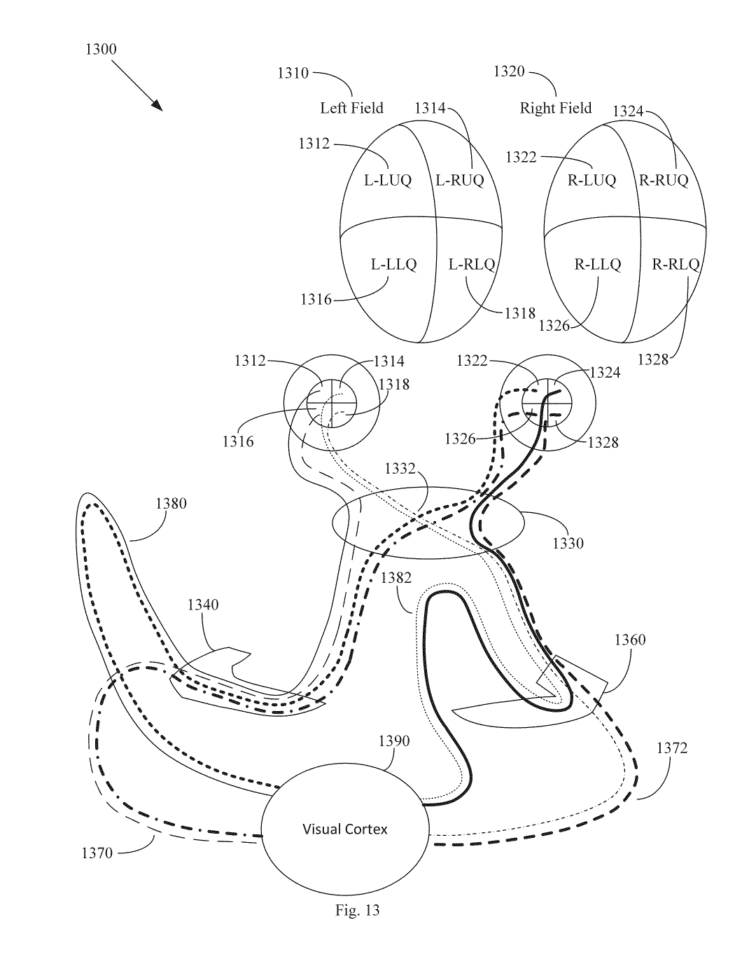

FIG. 13 illustrates a cortical representation of visual space used to represent mental desktop space provided by a BCI system 1300 according to an embodiment. The BCI system 1300 enables use of the mental desktop for access, navigation, command, and control of digital devices and content functions. Human-computer interfaces now available rely on the physical senses, e.g., vision, and physical movements, e.g., hands controlling a mouse or keyboard, to provide an interactive platform for accessing and controlling digital devices and content. These physical and perceptual inputs and controls are limited and restrict the potential expression of more novel and efficient human-computer interfaces. Existing systems that provide brain-computer interfaces are based on electroencephalography (EEG) technology and control functions from a predetermined library that define the outputs and leave the inputs open-ended. Moreover, the inputs are provide using spectral analysis of electrical activity, which is fundamentally different than the use of spatial and hemispherical information that mental gestures used in a visual BCI workspace according to an embodiment.

According to the embodiment described with respect to FIG. 13, a BCI system 1300 enables users to operate computing devices by focusing mental attention on different sections of the visual field. This visual field is referred to as the “mental desktop.” When users mentally visualize or attend to the upper-left quadrant of their visual field, an associated command may be executed. However, this does not mean the user changes eye focus to that area, but may simply think about, i.e., visualize, that area. The areas of the visual field have particularly strong mapping to brain activity measures.

FIG. 13 shows the mental desktop includes a left field 1310 and right field 1320. The left field 1310 is arranged to include a left-left upper quadrant 1312, a left-right upper quadrant 1314, a left-left lower quadrant 1316 and a left-right lower quadrant 1318. The right field 1320 is arranged to include a right-left upper quadrant 1322, a right-right upper quadrant 1324, a right-left lower quadrant 1326 and a right-right lower quadrant 1328.

Visual signals from the retina reach the optic chiasma 1330 where the optic nerves partially cross 1332. Images on the sides of each retina cross over to the opposite side of the brain via the optic nerve at the optic chiasma 1330. The temporal images, on the other hand, stay on the same side. This allows the images from either side of the field from both eyes to be transmitted to the appropriate side of the brain, combining the sides together. This allows for parts of both eyes that attend to the right visual field to be processed in the left visual system in the brain, and vice versa. The optic tract terminates in the left geniculate nucleus 1340 and right geniculate nucleus 1360. The left geniculate nucleus 1340 and right geniculate nucleus 1360 are the primary relay center for visual information received from the retina of the eye. The left 1340 and right 1360 geniculate nuclei receive information from the optic chiasma 1330 via the optic tract and from the reticular activating system. Signals from the left and right geniculate nucleus are sent through the optic radiations 1370, 1372, which act as a direct pathway to the primary visual cortex 1390. In addition, the left 1340 and right 1360 geniculate nuclei receive many strong feedback connections from the primary visual cortex. Meyer's loops 1380, 1382 are part of the optic radiation that exit the left lateral geniculate nucleus 1340 and right lateral geniculate nucleus 1360, respectively, and project to the primary visual cortex 1390. The visual cortex 1390 is responsible for processing the visual information.

For a “mental desktop,” eye tracking technology may be used to effect navigation and command and control of computer interfaces. However, eye tracking technology is constrained to the physical space and suffers from the same limitations as archetypal operating system models. For example, looking up and to the left has been used to translate a mouse pointer to that region.

Computer interfaces utilize a digital desktop space represented by physical space on a computer display. In contrast, a mental desktop according to an embodiment detaches the physical desktop into mental workspace, i.e., mental desktop that divides workspace into visuospatial regions based on regions of an individual's field of view referred to as visual hemifields.

Visual information is naturally segregated by the left and right eye as well as upper and lower, and left and right divisions, within the left and right eyes. These divisions create hemifields that are each represented in corresponding brain regions. The organization of the brain around the hemifields is referred to as retinotopic organization because regions of the retina are represented in corresponding brain regions.

The mental desktop's workspace facilitates access of assigned information, e.g., application shortcut, file, or menu, by looking or mentally visualizing a region in visual space. In summary, the mental desktop creates an imaginary desktop space for a user to use in a similar manner as a digital desktop in current operating systems.

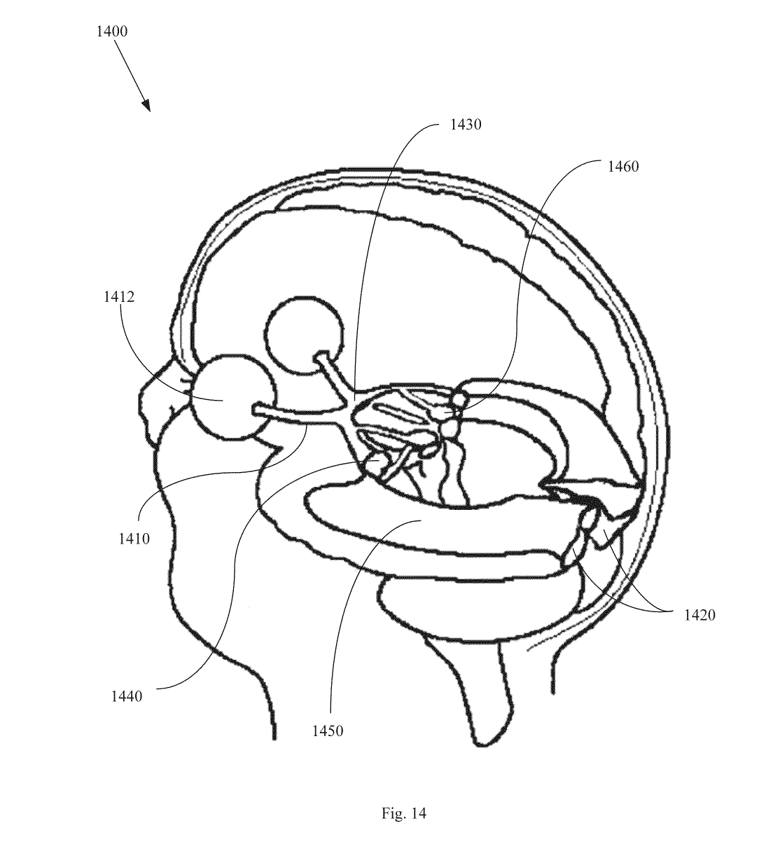

By extracting the eight visual hemifields through the retinotopic organization in the human primary visual cortex, a mental desktop may be implemented by the BCI system. FIG. 14 is a diagram of the optic system 1400 according to an embodiment. In FIG. 14, the optical nerve 1410 tracks from eyes 1412 to the human primary visual cortex 1420 in the occipital lobe of the brain.

The visual signals pass from the eyes 1412 and through the optic nerve 1410 to the optic chiasm. For example, looking at or visualizing the upper right visual field to the right of the midline produces concomitant brain activity in visual cortex corresponding to the same hemifield in the upper right of the right eye. The retinotopic organization of the visual cortex allows for the use of visuospatial information decoded from brain activity into usable information for mental desktop to identify the region a user wishes to access. As described above, images on the sides of each retina cross over to the opposite side of the brain via the optic nerve 1410 at the optic chiasma 1430. The temporal images, on the other hand, stay on the same side. The lateral geniculate nuclei 1440 (left and right) are the primary relay center for visual information received from the eye 1412.

The signals are sent from the geniculate nuclei 1440 through the optic radiations 1450. The optic radiations 1450 are the direct pathway to the primary visual cortex 1420. Unconscious visual input goes directly from the retina to the superior colliculus 1460.

Table 1 illustrates the mapping of the left and right field quadrants to the visual cortex through the optic chiasma, left and right geniculate nucleus, and the optic radiations including the Meyer's loop.

TABLE 1

| | Directly Through | | Optic Radiation to |
|---|---|---|---|
| | Optic Chiasma or | Left or Right | Visual Cortex or |
| Field | Cross-Over in Optic | Geniculate | Meyer's Loop to |
| Quadrant | Chiasma | Nucleus | Visual Cortex |
| Left Field | | | |
| L-LUQ | OC | LGN | OR2VC |
| L-RUQ | OCX | RGN | OR2VC |
| L-LRLQ | OC | LGN | ML2VC |
| L-RLQ | OCX | RGN | ML2VC |
| Right Field | | | |
| R-LUQ | OCX | LGN | OR2VC |
| R-RUQ | OC | RGN | OR2VC |
| R-LLQ | OCX | LGN | ML2VC |
| R-RLQ | OC | RGN | ML2VC |

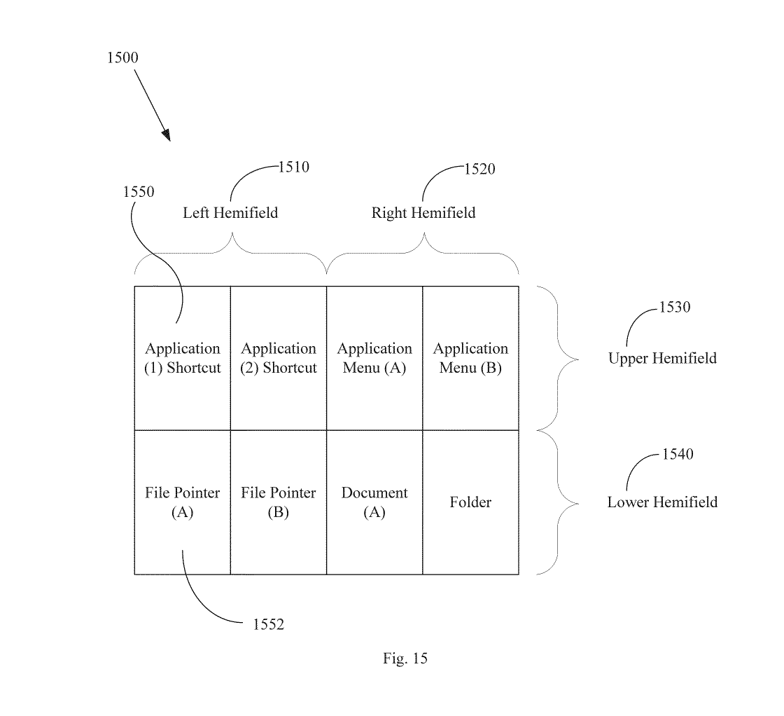

FIG. 15 shows the physiologically segregated sections of visual space 1500 according to an embodiment. The BCI system assigns content to physiologically segregated sections of visual space. In FIG. 15, the visual space 1500 is organized into a left hemifield 1510 and right hemifield 1520. The visual space 1500 is also arranged into an upper hemifield 1530 and a lower hemifield 1540. Thus, according to an embodiment, the visual space 1500 may be assigned content, which the user may implement based on visualization of the appropriate area in the visual space 1500. Content may be anything, such as application shortcuts, file pointers, files, etc. The user would simply look at or visualize a particular hemifields to access whatever content is assigned to the location that is seen or visualized according to that physiologically segregated section of visual space. Each of the eight hemifields of the visual space 1500 may have content assigned to the region in space accessed through visualization or any activity in the hemifield space. For example, a first application shortcut 1550 may be assigned to the left-left upper hemifield. A file pointer 1552 may be assigned to the left-left lower hemifield.

A training system may be utilized to map each individual's visual field to define the regions of visual space 1500 in FIG. 15 to correspond to regions of the visual field. The acuity of the mental desktop may be refined with spatial boundaries in the visual field. Further, users may use a head mounted display to facilitate the visualization of the division of visual space 1500 available on the mental desktop. An individual's mental desktop may be remotely stored, e.g., cloud storage, to enable the mental desktop workspace on any device.

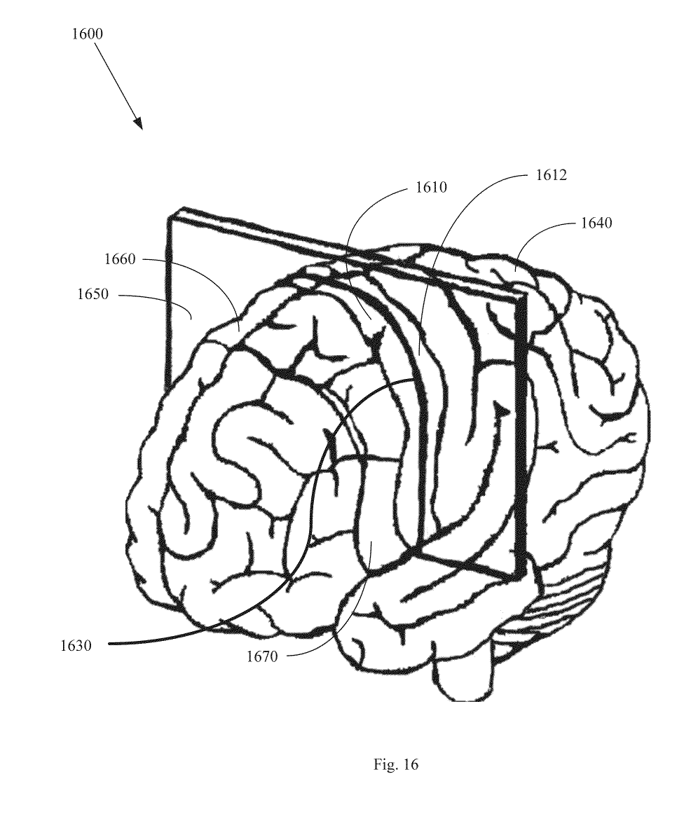

FIG. 16 is a model of a human cortex 1600 showing the primary motor cortex 1610 and the primary sensory cortex 1612 as utilized by a BCI system according to an embodiment. The primary motor cortex 1610 and the primary sensory cortex 1612 are anterior and posterior to the central sulcus 1630, respectively, indicated by the vertical slice. The central sulcus 1630 is a fold in the cerebral cortex, which represents a prominent landmark of the brain that separates the parietal lobe 1640 from the frontal lobe 1650 and the primary motor cortex 1610 from the primary somatosensory cortex 1660.

According to an embodiment, mental gestures are mapped to brain activity emerging from topographically organized brain regions, such as the primary motor and/or somatosensory cortices found in the human brain. These two brain areas each have regions that are divided into discrete areas that are dedicated to controlling corresponding body locations. In FIG. 16, the supplementary motor cortex 1660 is shown generally midline of the frontal lobe 1650. The supplementary motor cortex 1660 is the part of the cerebral cortex 1600 that contributes to the control of movement. The primary motor cortex 1610 is located in the posterior portion of the frontal lobe 1650. The primary motor cortex 1610 is involved with the planning and execution of movements. The posterior parietal cortex 1640 receives input from the three sensory systems that play roles in the localization of the body and external objects in space: the visual system, the auditory system, and the somatosensory system. The somatosensory system provides information about objects in our external environment through touch, i.e., physical contact with skin, and about the position and movement of our body parts through the stimulation of muscle and joints. In turn, much of the output of the posterior parietal cortex 1640 goes to areas of frontal motor cortex 1670. The premotor cortex 1670 lies within the frontal lobe 1650 just anterior to the primary motor cortex 1610. The premotor cortex 1670 is involved in preparing and executing limb movements and coordinates with other regions to select appropriate movements.

User interfaces that utilize physical or perceptual inputs use a set of protocol to perform pre-determined actions. For example, a keyboard uses keys that have characters assigned to each or a mouse uses X-Y locations and clicks to indicate a response. Similarly, a BCI system needs a foundation to establish a widespread, practical use of BCI inputs. According to an embodiment, a BCI system implements and processes mental gestures to perform functions or provide other types of input. Mental gestures are a library of thought gestures interpreted from brain activity to be used as a computer input in the same way a keys on a keyboard provide pre-determined input for flexible control over their output.

For example, touch-enabled surfaces have pre-set gestures such as pinching, squeezing, and swiping. These touch gestures serve as a foundation to build touch interfaces and usages across tasks. Similarly, mental gestures follow the same principle of establishing a foundation for BCI input through a library of BCI gestures, i.e., mental gestures, to enable usages across tasks and even platforms.

Mental gestures are executable through thought and recorded directly from brain activity. In contrast to touch gestures that are based on actual movement, mental gestures are imagined motor movements. The combination of a library of mental gestures and the flexibility of using a wide number of imagined movements rather than a single modality such as touch present the potential for an extremely powerful interface to a BCI system. Benefit to mental gestures over traditional inputs include (1) the user doesn't need to physically input any information, which would allow people without limbs or control of those limbs to perform the actions, (2) the mental gestures may emerge from any imagined motor movements that would not be practical as physical inputs, e.g., kicking, (3) the range of possible mental gestures expands the flexibility and utility over traditional inputs such as mice, keyboards, and trackpads that rely on manual inputs, and (4) mental gestures may be hemisphere specific because many brains have a left and right lateralized brain hemispheres that may create independent motor signals.

Examples of mental gestures include but are not limited to single digit movement, digit movement of different numbers, e.g., 1, 2 or 3-finger movement), hand waving, kicking, toe movement, blinks, head turning, hand nodding, bending at the waist, etc. The movements represented by mental gestures are purely imagined movements that may be associated with a variety of computer inputs. For example, an operating system may assign functionality to single-digit movement and a different functionality to two-digit movement. Alternatively, a media player could assign each of its functions, e.g., play/pause, reverse, shuffle, etc., to different mental gestures.

One possible implementation of mental gestures would be a software development kit (SDK) with a library of mental gestures for developers to assign to proprietary functions within their software. A SDK is a set of software development tools that allows for the creation of applications for a system. The SDK would enable developers to access mental gestures that may be used in a flexible, open-ended way. For example, a videogame developer could use the mental gestures SDK to develop BCI control over aspects of a videogame or a mobile original equipment manufacturer (OEM) could use the mental gestures SDK to develop mental gesture control over proprietary functions on their mobile device.

Mental gestures could also be used with another system that could combine multiple sources of inputs. If a cross-modal perceptual computing solution existed, mental gestures may be an additional source of input to be combined with other perceptual inputs. For example, air gestures could combine with mental gestures to code for left or right-handed air gestures based on left-lateral or right-lateral mental gesture input.

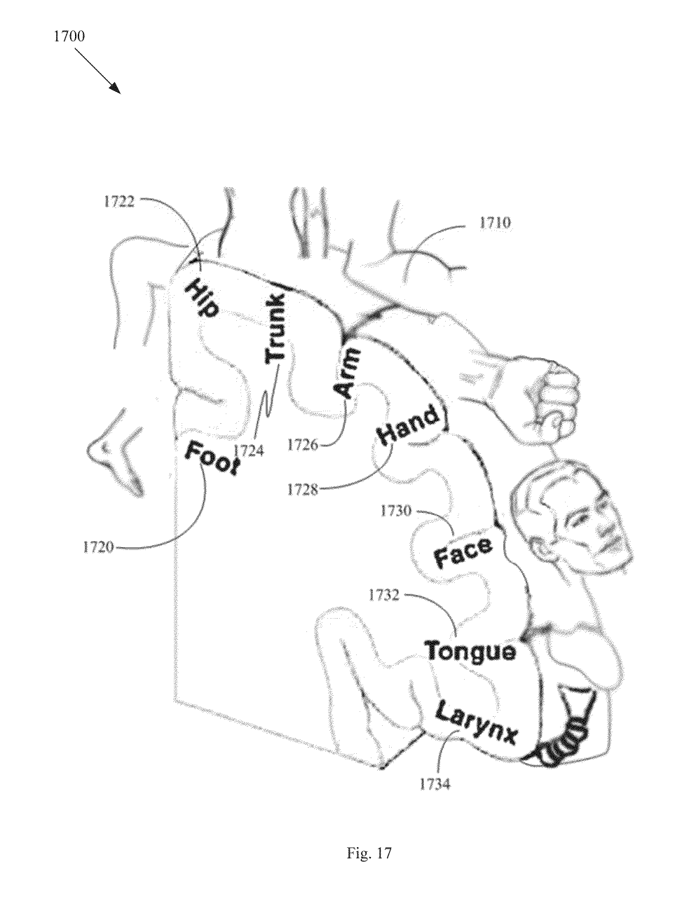

FIG. 17 is a model 1700 demonstrating the topographical organization of the primary motor cortex 1710 on the pre-central gyrus of the human cortex. Each part of the body is represented by distinct areas of the cortex with the amount of cortex associated with the control of its respective body part. FIG. 17 shows the areas responsible for preparation and execution of movement of the foot 1720, the hip 1722, the trunk 1724, the arm 1726, the hand 1728, the face 1730, the tongue 1732 and the larynx 1734.

Any brain imaging device with spatial resolution high enough to extract the signals from a segment of cortex narrow enough to distinguish between neighboring areas may be used to implement the mental desktop. Some examples of currently available devices include dense electrode EEG, fNIRS, MRI, or MEG.

For each signal from the brain, the hemisphere (left or right), spatial location, and area are responsible for codes for the source of the motor signal. For example, activity or imagined activity of the left index finger would produce activity in the finger area in the right hemisphere. Mental gestures would code for left, single digit movement and the location and amount of area active would code for the precise finger and the number of digits, i.e., 1, 2, 3, or 4-digit gestures.

Thus, a system for implement a mental desktop that uses mental gestures for input according to an embodiment may include neuroimaging devices, such as fNIRS, EEG. MEG. MRI, ultrasound, etc. Methodologies for obtaining or analyzing brain activity may also include modified Beer-Lambert Law, event related components, multi-voxel pattern analysis, spectral analysis, use of MVPA on fNIRS.

According to an embodiment, a BCI system provides a mental desktop that maps computer content and functions to different sections of the visual field. The BCI system allows users to be trained in the application of the above-referenced system. A library of thought gestures that are interpreted from brain activity may be used to affect computer navigation, command, and control. Further, development systems may be provided to allow software developers to utilize mental gestures.

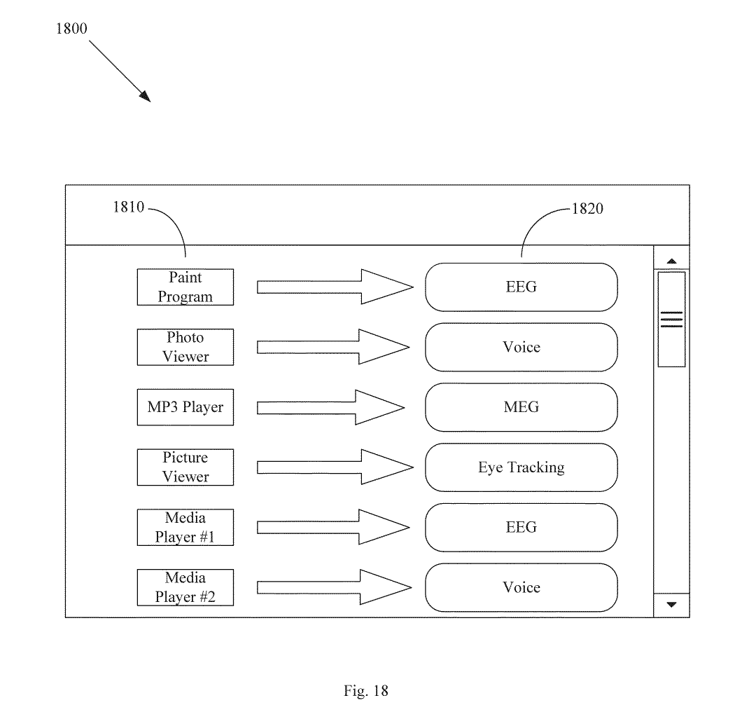

FIG. 18 illustrates a user interface 1800 for assigning BCI measures and other modalities to applications according to an embodiment. In FIG. 18, a column of applications 1810 are shown on the left. BCI measures and other modalities 1820 are shown on the right. Other applications 1810 as well as other BCI measures and other modalities 1820 may be implemented. The BCI measures and other modalities 1820 may be selected for assignment to at least one of the applications 1810 on the left. Thus, a BCI system according to an embodiment may be used in multimodal systems.

By combining BCI with other modalities, e.g., gesture, voice, eye tracking, and face/facial expression tracking, new user experiences and ways for users to control electronic devices may be provided. Thus, the BCI system according to an embodiment recognizes both BCI types of input as well as other modalities. In addition, some approaches to feedback loops with brain activity elicitation may be implemented, and contextual sensing may alter the use of BCI input.

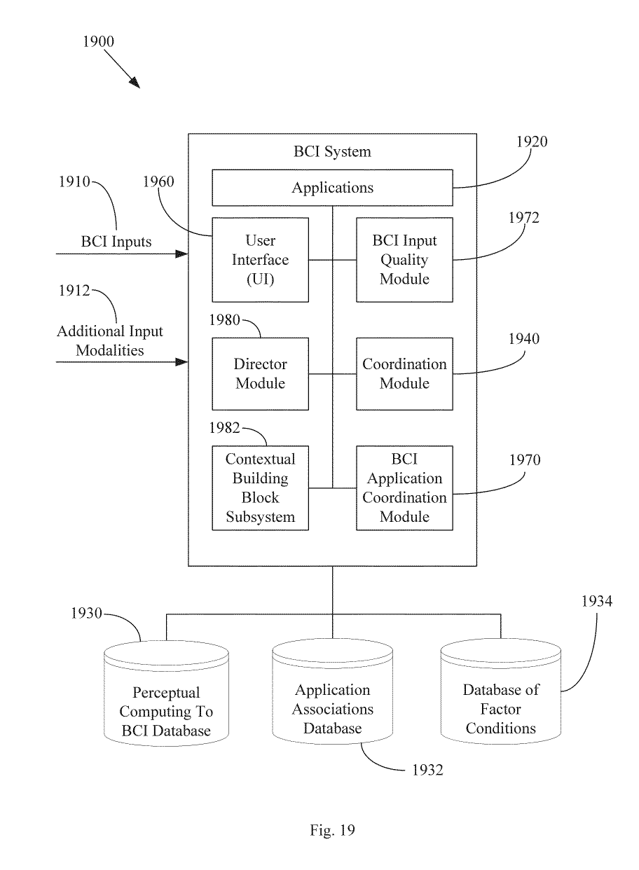

FIG. 19 shows a BCI system 1900 that accepts BCI inputs 1910 and other modality inputs 1912 according to an embodiment. In FIG. 19, BCI inputs 1910 are provided to the BCI system 1900 for implementation with applications 1920. Additional modalities 1912 are also provided to the BCI system 1900 for implementation with applications 1920. However, some of the BCI inputs 1910 and the additional modalities 1912 may be mutually exclusive while some may be used together. A perceptual computing to BCI database 1930 may be used to hold heuristics on how natural UI inputs and BCI inputs work together. A coordination module 1940 receives input from BCI inputs 1912, the additional input modalities 1912 and perceptual computing inputs from the database 1930 and then makes determinations on the determined user intent. The coordination module 1940 makes a final determination of user intent based on results from the BCI application coordination module 1970 and initiates a final command. A UI 1960, as shown in FIG. 19, may be used for assigning the BCI 1910 and additional modality 1912 inputs to applications 1920. An application associations database 1932 may be used to store BCI/application associations. A BCI application coordination module 1970 monitors whether assigned applications are running and initiates BCI control for relevant applications.

A BCI input quality module 1972 monitors environmental signals that degrade sensor input. The BCI system further includes a factor database of factor conditions 1934, which includes the variables described above and their levels that inhibit particular forms of input 1910, 1912. A director module 1980 receives the inputs 1910, 1912, weighs them against the factor database 1934, and sends commands to the applications 1920 to control how the inputs 1910, 1912 are used, e.g., turned off, turned on, some measures weighed more than others, etc. A contextual building block subsystem 1982 measures environmental and user factors. A determination is made by the director module 1980 whether possible interference is occurring. If interference is detected, the director module 1980 adjusts the BCI input 1910.

With input modalities such as hand gestures, voice, and eye-tracking, one challenge is alerting the system to an imminent command through one of those modalities, e.g., a voice command. The system may interpret inadvertent noise or movements, before or after the actual command, as a command. A BCI pattern from the user immediately before the command could signal that the next major sensor-detected event may be interpreted as the command.

When a system is subject to input from one or more modalities at the same time, BCI input could indicate which modality is to have precedence. One example of the use of cross-modal BCI input would be the use of BCI input to determine whether it is a gesture is a right or left handed gesture base. Alternatively, BCI input may be used simultaneously with another modality to reinforce the input command. For example, a brain activity pattern may be measured at the same time as a voice command. The brain activity pattern may be used to help the system differentiate between 2 similar sounding commands.

BCI systems according to an embodiment that include life blogging and “total recall” systems that records audio and video from the wearer's point of view may be used to aid people with cognitive deficits. Software algorithms may be used determine aspects of the sensor input. For example, an elderly person with memory loss could wear such a device, and when the BCI detects a confused state, through electrical patterns and/or blood flow, the system could give audio information in the earpiece that reminds the user of the names of people and objects in view.

See and think commands and tracking may be provided. A user could use eye tracking input to select a target, and then use the BCI system to provide input to act on the target that is being focused upon. e.g., the object the user is looking at changes color based on the brain activity pattern. This example could also be applied to visual media, e.g., the user could focus on a character, and the user's brain activity pattern could mark that character as being more interesting. Further, as a user reads, confusion may be detects to indicate the text was not understood, which may be helpful in teaching.

BCI input may be used to address cross modality interruption. The BCI input may be used to interrupt a system that is responding to another modality. For example, in a game, a user may use an air gesture to move a character in a direction, then use a change in BCI input to stop the character. UI feedback may also be used with BCI input. For example, a BCI system may provide various feedback to users when the system identifies BCI input, allowing the user to know the input has been received and confirmed. The BCI feedback could occur with other modality feedback, such as gesture. Further, a UI may be used for user mapping of BCI input. A user interface allows a user to map brain activity patterns to a given modality so that the system activates a command window-of-opportunity for that modality when the corresponding BCI pattern occurs. A user may map brain activity patterns to a given modality, so that the system has higher reliability in recognizing a command because the sensed inputs correlate to a brain activity pattern plus another modality. A user may also map different modalities to different brain activity patterns so that one pattern will mean that a correlating modality may be active, while another pattern activates a different modality.

BCI input may also be used to activate system resources. For example, a system may be alerted to come out of power state when the user becomes more alert. This may be used when a user is doing visual design. The BCI system could allow a processor to go into a sleep state as the user is in browsing mode. When brain activity patterns indicate that the user is about to take action, such as making an edit, the system could power up the processor so the processor is more responsive when the user starts the action.

According to an embodiment, the BCI system 1900 enables users to assign BCI input to one application that may not have focus, wherein focus refers to the application that currently has the attention of the OS. An application would then respond to BCI input even though the user is doing something else. A UI enables the user to assign the application.

Other examples of embodiments may include music and audio implementations where BCI input is accepted for control while the user is editing a document. Communication channels may show the user's status, e.g., busy thinking, through an instant messaging (IM) client while the user is being productive. Particular brain regions facilitate switching between tasks and BCI input to change the music may facilitate switching between tasks. The BCI system may be mapped to a music player so that whenever the task-switching portion of the brain becomes active, the music player skips to the next track to facilitate switching to a new task. In addition, autonomous vehicles will allow drivers to escape the demands of driving to enjoy non-driving activities in a vehicle. However, when the duties of driving return to the driver, the non-driving activities withdraw. The BCI system may map entertainment features of an in-vehicle infotainment system to cognitive workload to switch off entertainment features when a certain workload level is reached.

The BCI system could also make determinations about user context in order to allow various BCI inputs to be used at a given time. A status indicator could show the user when BCI input is available as an input. Other contextual determinations may be provided by the BCI system according to an embodiment. For example, the activity of the user may be determined by biometric sensors measuring as heart rate, respiration, movement, by accelerometers and gyroscopes, and user position, e.g., standing up versus lying down. At certain activity levels, unreliable BCI input may be prevented from being used by the system, or the system could adjust to the varying circumstances. The BCI system may determine whether the user is engaged in conversation and that information may be used as BCI input. BCI input for making contextual determinations may also include environmental conditions inhibiting reliable BCI input that causes user distraction, including sounds, visual stimuli, unpredictable noise, odor, media being played, and other factors and environmental conditions that could inhibit accurate measures due to electrical interference, such as magnetic fields, ambient temperature, and other environmental factors.

Different types of brain activity sensors have different strengths and benefits for a given task that the user is doing. For example, in instances where higher spatial resolution desired, the system may select fNIRS input rather than EEG which has lower spatial resolution. In other instances, rapid feedback may be desired so the system may select EEG or another technology that also has higher temporal resolution. Environmental sensors could determine user activities to influence which BCI input is best. Environmental factors such as electromagnetic energy are known to be detectable by EEG. In instances where electromagnetic (EM) energy would interfere with EEG recording, the BCI system may switch to a superior input source.

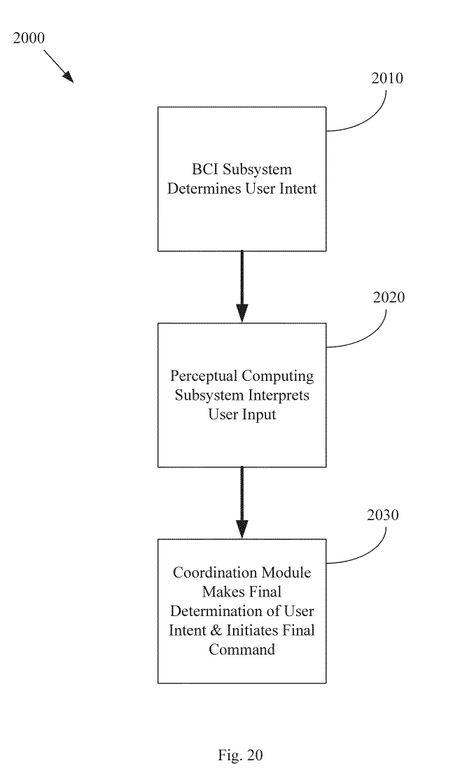

FIG. 20 illustrates a flowchart of a method 2000 for determining user intent according to an embodiment. A BCI system determines user intent 2010. A perceptual computing system interprets the user input 2020. A coordination module then makes a final determination of user intent and initiates final command 2030.

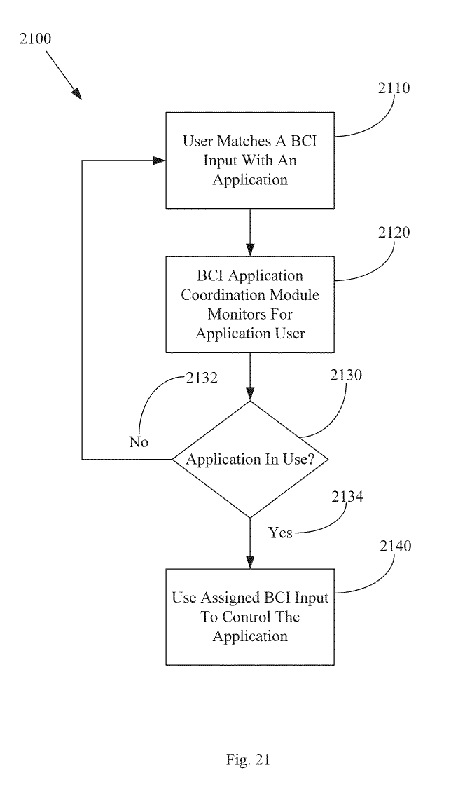

FIG. 21 is a flowchart of a method 2100 for assigning BCI input for controlling an application according to an embodiment. A user matches a BCI input with an application 2110. The BCI application coordination module monitors for application use 2120. A determination is made whether the application is in use 2130. If the application is not in use 2132, the process returns to match BCI input with an application. If the application is in use 2134, the assigned BCI input is used to control the application 2140.

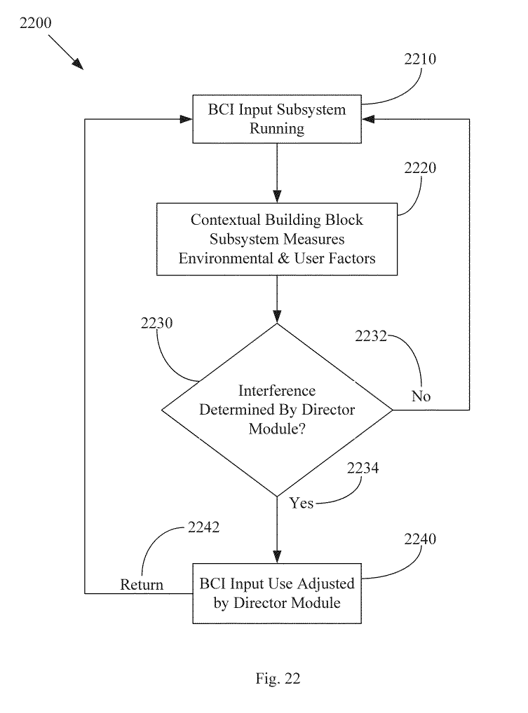

FIG. 22 is a flowchart of a method 2200 for adjusting contextual factors by the BCI system according to an embodiment. A BCI input subsystem is running 2210. The contextual building block subsystem measures environmental and user factors 2220. A determination is made by the director module whether possible interference is occurring 2230. If no, then at 2232 the process returns to the beginning of the process. If possible interference is detected 2234, the director module adjusts the BCI input 2240. The process may return to the beginning 2242.

Thus, according to embodiments described herein, brain/skull anatomical characteristics, such as gyrification, cortical thickness, scalp thickness, etc., may be used for identification/authentication purposes. Measured stimuli/response brain characteristics, e.g., anatomic and physiologic, may be translated into specific patterns to categorize a brain for identify/authentication purposes. The anatomical and physiologic brain data may be coupled to determine identity and authenticity of a user. Information on other brain signatures, e.g., anatomic and physiologic, and comparisons to similar brains may be used to predict a brain response to a new stimulus and for identification and/or authentication purposes. Brain identification and/or authentication techniques in combination with other identification and/or authentication techniques, e.g., password, other biometric parameters, may be used to increase the identity/authentication sensitivity and specificity.

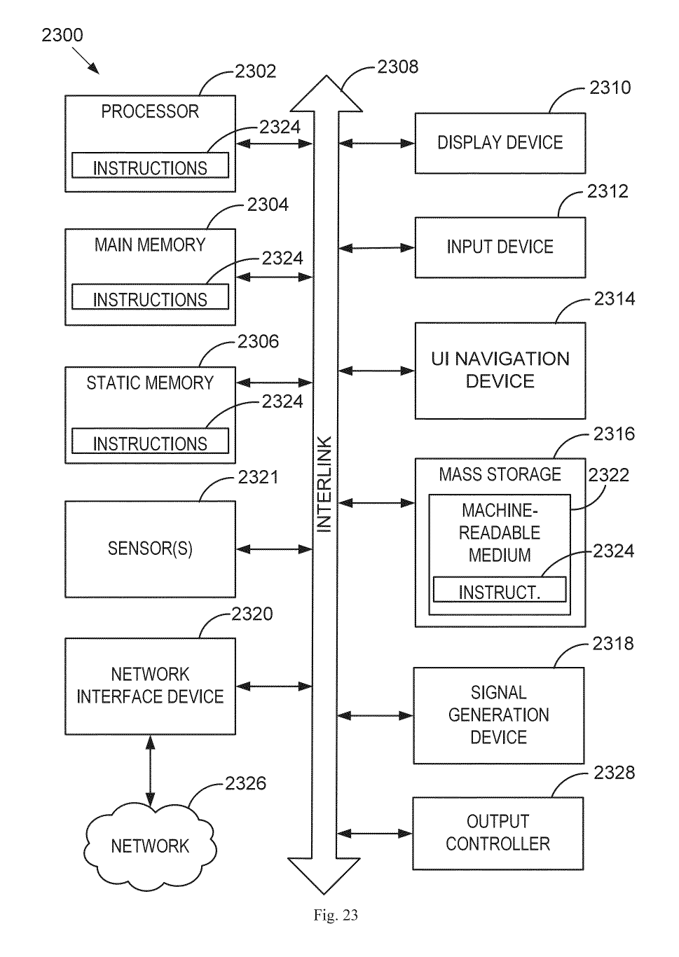

FIG. 23 illustrates a block diagram of an example machine 2300 for providing a brain computer interface (BCI) system based on gathered temporal and spatial patterns of biophysical signals according to an embodiment upon which any one or more of the techniques (e.g., methodologies) discussed herein may perform. In alternative embodiments, the machine 2300 may operate as a standalone device or may be connected (e.g., networked) to other machines. In a networked deployment, the machine 2300 may operate in the capacity of a server machine and/or a client machine in server-client network environments. In an example, the machine 2300 may act as a peer machine in peer-to-peer (P2P) (or other distributed) network environment. The machine 2300 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, at least a part of one or more computer systems (e.g., a standalone, client or server computer system) or one or more hardware processors 2302 may be configured by firmware or software (e.g., instructions, an application portion, or an application) as a module that operates to perform specified operations. In an example, the software may reside on at least one machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform at least part of any operation described herein. Considering examples in which modules are temporarily configured, a module need not be instantiated at any one moment in time. For example, where the modules comprise a general-purpose hardware processor 2302 configured using software; the general-purpose hardware processor may be configured as respective different modules at different times. Software may accordingly configure a hardware processor, for example, to constitute a particular module at one instance of time and to constitute a different module at a different instance of time. The term “application,” or variants thereof, is used expansively herein to include routines, program modules, programs, components, and the like, and may be implemented on various system configurations, including single-processor or multiprocessor systems, microprocessor-based electronics, single-core or multi-core systems, combinations thereof, and the like. Thus, the term application may be used to refer to an embodiment of software or to hardware arranged to perform at least part of any operation described herein.

Machine (e.g., computer system) 2300 may include a hardware processor 2302 (e.g., a central processing unit (CPU), a graphics processing unit (GPU), a hardware processor core, or any combination thereof), a main memory 2304 and a static memory 2306, at least some of which may communicate with others via an interlink (e.g., bus) 2308. The machine 2300 may further include a display unit 2310, an alphanumeric input device 2312 (e.g., a keyboard), and a user interface (UI) navigation device 2314 (e.g., a mouse). In an example, the display unit 2310, input device 2312 and UI navigation device 2314 may be a touch screen display. The machine 2300 may additionally include a storage device (e.g., drive unit) 2316, a signal generation device 2318 (e.g., a speaker), a network interface device 2320, and one or more sensors 2321, such as a global positioning system (GPS) sensor, compass, accelerometer, or other sensor. The machine 2300 may include an output controller 2328, such as a serial (e.g., universal serial bus (USB), parallel, or other wired or wireless (e.g., infrared (IR)) connection to communicate or control one or more peripheral devices (e.g., a printer, card reader, etc.).

The storage device 2316 may include at least one machine readable medium 2322 on which is stored one or more sets of data structures or instructions 2324 (e.g., software) embodying or utilized by any one or more of the techniques or functions described herein. The instructions 2324 may also reside, at least partially, additional machine readable memories such as main memory 2304, static memory 2306, or within the hardware processor 2302 during execution thereof by the machine 2300. In an example, one or any combination of the hardware processor 2302, the main memory 2304, the static memory 2306, or the storage device 2316 may constitute machine readable media.

While the machine readable medium 2322 is illustrated as a single medium, the term “machine readable medium” may include a single medium or multiple media (e.g., a centralized or distributed database, and/or associated caches and servers) that configured to store the one or more instructions 2324.

The term “machine readable medium” may include any medium that is capable of storing, encoding, or carrying instructions for execution by the machine 2300 and that cause the machine 2300 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having resting mass. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 2324 may further be transmitted or received over a communications network 2326 using a transmission medium via the network interface device 2320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks ((e.g., channel access methods including Code Division Multiple Access (CDMA), Time-division multiple access (TDMA), Frequency-division multiple access (FDMA), and Orthogonal Frequency Division Multiple Access (OFDMA) and cellular networks such as Global System for Mobile Communications (GSM), Universal Mobile Telecommunications System (UMTS), CDMA 2000 1×*standards and Long Term Evolution (LTE)), Plain Old Telephone (POTS) networks, and wireless data networks (e.g., Institute of Electrical and Electronics Engineers (IEEE) 802 family of standards including IEEE 802.11 standards (WiFi), IEEE 802.16 standards (WiMax®) and others), peer-to-peer (P2P) networks, or other protocols now known or later developed.

For example, the network interface device 2320 may include one or more physical jacks (e.g., Ethernet, coaxial, or phone jacks) or one or more antennas to connect to the communications network 2326. In an example, the network interface device 2320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 2300, and includes digital or analog communications signals or other intangible medium to facilitate communication of such software.

### Additional Notes & Examples

Example 1 may include subject matter (such as a device, apparatus, client or system) including a library of stimuli for provisioning to a user, a data collection device for gathering temporal and spatial patterns of biophysical signals associated with brain activity in response to provisioning stimuli from the library of stimuli to the user and a processing device for correlating the gathered temporal and spatial patterns of biophysical signals associated with brain activity to identify a brain signature of the user and performing a processor controlled function based on the brain signature of the user identified through correlating the gathered temporal and spatial patterns of biophysical signals associated with brain activity.

Example 2 may optionally include the subject matter of Example 1, wherein the processing device compares mental profile of the user derived from the brain signature of the user with mental profiles from a database of mental profiles of a predetermined population.

Example 3 may optionally include the subject matter of any one or more of Examples 1-2, wherein the processing device calculates statistics and probability of a match of the mental profile of the user for any of a range of topics.

Example 4 may optionally include the subject matter of any one or more of Examples 1-3, wherein the processing device builds a mental profile of the user as a function of the stimuli based on the temporal and spatial patterns of biophysical signals associated with brain activity of the user.

Example 5 may optionally include the subject matter of any one or more of Examples 1-4, wherein the processing device combines the brain signature of the user with personal data and other traits obtained from a database to develop a mental profile model of the user.

Example 6 may optionally include the subject matter of any one or more of Examples 1-5, wherein the processing device correlates probabilities between subjects and calculates statistics and probability of a mental match between the mental profile model of the user and mental profile models of at least one other user.

Example 7 may optionally include the subject matter of any one or more of Examples 1-6, wherein the processing device provides identification and authentication of a user, wherein a mental profile of a user is created by the processing device during a calibrating stage based on presentation of stimuli from the library of stimuli to the user, the processing device further determining whether a mental profile of a user being authenticated correlates to the mental profile of the user created during the calibration stage.

Example 8 may optionally include the subject matter of any one or more of Examples 1-7, wherein the processing device is arranged to perform telepathic contextual search by monitoring transmissions from a brain-computer interface system of a user, displaying stimuli associated with brain activity measurements from the user, searching for the brain activity measurements to locate a search object associated with the brain activity measurements and returning search results based on a match between the brain activity measurements and search objects having the associated brain activity measurements correlated therewith.

Example 9 may optionally include the subject matter of any one or more of Examples 1-8, wherein the processing device provides telepathic augmented reality by receiving input from brain-computer interface (BCI) sensors and detectors and a biometric and environmental sensor array, the processing device arranged to map input and data obtained a database of recognized sensor inputs, AR character and AR environmental content to an AR experience, the processing device blending AR characters with the environment and presenting the AR experience to a user based on the user intent derived from the input from the brain-computer interface (BCI) sensors and detectors and the biometric and environmental sensor array.

Example 10 may optionally include the subject matter of any one or more of Examples 1-9, wherein the processing device creates a mental desktop representing a left and right hemifield for each of a left and right eyes of a user, the processing device further segregating each eye into an upper division and a lower division, wherein the mental desktop includes eight areas of a visual field of the user having information assigned thereto, the processing device detecting mentally visualization of a region in the mental desktop and implementing a function according to the information assigned to the mentally visualized region.

Example 11 may optionally include the subject matter of any one or more of Examples 1-10, wherein the processing device is arranged to analyze receive inputs including temporal and spatial patterns of biophysical signals associated with brain activity of the user and additional input modalities received for implementation with applications, and perceptual computing inputs from the perceptual computing to BCI database, the processing device further arranged to determine an intent of the user based on the inputs and interrelatedness data associated with the inputs obtained from a perceptual computing database and factors obtained from a factor database, wherein the processing device initiates a command based on the determined user intent.

Example 12 may optionally include the subject matter of any one or more of Examples 1-11, wherein the processing device is arranged to determine whether interference is occurring and to adjust the wherein the temporal and spatial patterns of biophysical signals of the user to account for the interference.

Example 13 may optionally include the subject matter of any one or more of Examples 1-12, further includes a user interface for assigning temporal and spatial patterns of biophysical signals associated with brain activity of the user and additional modality inputs to applications.

Example 14 may include or may optionally be combined with the subject matter of any one of Examples 1-13 to include subject matter (such as a method or means for performing acts) for providing stimuli to a user, gathering temporal and spatial patterns of biophysical signals associated with brain activity in response to providing the stimuli to the user, correlating the gathered temporal and spatial patterns of biophysical signals associated with brain activity to identify user brain signatures and performing a processor controlled function based on the user brain signatures identified through correlating the gathered temporal and spatial patterns of biophysical signals associated with brain activity.

Example 15 may optionally include the subject matter of Example 14, wherein the processor controlled function includes determining at least one similarity between identified patterns of the user and patterns common to a group of users.

Example 16 may optionally include the subject matter of any one or more of Examples 14-15, further includes providing a user with a brain monitoring device and running the user through a series of experiences associated with the stimuli, wherein the correlating the gathered temporal and spatial patterns includes characterizing the gather spatial and temporal brain activity patterns to identify the user brain signatures.

Example 17 may optionally include the subject matter of any one or more of Examples 14-16, wherein the performing a processor controlled function further includes building a characteristic mental profile of the user based on the user brain signatures, establishing models of mental predilections and personality traits and using the established models to predict an affinity of the user with an association of people.

Example 18 may optionally include the subject matter of any one or more of Examples 14-17, wherein the correlating the gathered temporal and spatial patterns of biophysical signals further includes translating recorded brain activity patterns in response stimuli into a characteristic mental profile associated with the stimuli, maintaining mental profiles to the stimuli for each individual in a database, integrating personal data and traits into the mental profiles, identifying a mental match between the mental profile of the user in response to the stimuli and at least one mental profile of other users associated with the stimuli and providing a probabilistic or percentage score of a mental match.

Example 19 may optionally include the subject matter of any one or more of Examples 14-18, wherein the providing stimuli to a user, gathering temporal and spatial patterns of biophysical signals associated with brain activity in response to providing the stimuli to the user and correlating the gathered temporal and spatial patterns of biophysical signals further includes calibrating a brain signature of a user based on the stimuli and authenticating the user by comparing a currently measured brain signature and the calibrated brain signature.

Example 20 may optionally include the subject matter of any one or more of Examples 14-19, wherein the calibrating the brain signature of the user includes presenting a set of stimuli to a user to incite brain activity responses, measuring brain anatomy and activity in response to the presented set of stimuli, performing pattern recognition of the measurements of brain anatomy and activity to produce a brain signature of the user, storing the produced brain signature and adding the stored brain signature to a database of anatomical and physiologic brain signatures of a predetermined population.

Example 21 may optionally include the subject matter of any one or more of Examples 14-20, wherein the presenting a set of stimuli further includes running the user through thoughts to incite certain characteristic brain activities.

Example 22 may optionally include the subject matter of any one or more of Examples 14-21, wherein the running the user through thoughts includes running the user through one selected from a group consisting of a series of memorized thoughts, patterns of muscle activation and imagined activities.

Example 23 may optionally include the subject matter of any one or more of Examples 14-22, wherein the measuring brain anatomy and activity includes measuring brain anatomy and activity using at least one of functional near infrared spectroscopy, electroencephalography, magnetoencephalography, magnetic resonance imaging and ultrasound.

Example 24 may optionally include the subject matter of any one or more of Examples 14-23, wherein the measuring brain anatomy and activity includes measuring anatomical characteristics.

Example 25 may optionally include the subject matter of any one or more of Examples 14-24, wherein the measuring anatomical characteristics includes measuring at least one of gyrification, cortical thickness and scalp thickness.

Example 26 may optionally include the subject matter of any one or more of Examples 14-25, wherein the performing pattern recognition of the measurements of brain anatomy and activity further includes performing pattern recognition based on at least one of modified Beer-Lambert Law, event-related components, multi-voxel pattern analysis (MVPA), spectral analysis, and MVPA on fNIRS.

Example 27 may optionally include the subject matter of any one or more of Examples 14-26, wherein the performing pattern recognition of the measurements of brain anatomy and activity further includes translating anatomic and physiologic measurements into specific patterns that can be used to categorize a brain for identification and authentication.

Example 28 may optionally include the subject matter of any one or more of Examples 14-27, wherein the authenticating the user includes presenting a previously applied set of stimuli to a user to incite brain activity responses, measuring brain anatomy and activity of the user based on the previously applied set of stimuli, performing pattern recognition of the measurements of brain anatomy and activity to produce a brain signature of the user and analyzing the brain signature of the user obtained through the performing the pattern recognition by comparing the brain signature with the calibrated brain signature of the user.

Example 29 may optionally include the subject matter of any one or more of Examples 14-28, wherein the analyzing the brain signature of the user includes comparing the brain signature with anatomical and physiologic brain signatures of a predetermined population.

Example 30 may optionally include the subject matter of any one or more of Examples 14-29, wherein the analyzing the brain signature of the user includes comparing the brain signature with additional identification and authentication techniques to increase sensitivity and specificity of the identification and authentication techniques.

Example 31 may optionally include the subject matter of any one or more of Examples 14-30, wherein the comparing the brain signature with additional identification and authentication techniques includes comparing the brain signature with at least one of handwriting recognition results, a password query and an additional biometric parameter.

Example 32 may optionally include the subject matter of any one or more of Examples 14-31, wherein the performing a processor controlled function based on the user brain signatures includes directing a device to perform a function in response to the gathered temporal and spatial patterns of biophysical signals associated with the brain activity.

Example 33 may optionally include the subject matter of any one or more of Examples 14-32, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes presenting a set of stimuli to a user, obtaining brain-computer interface (BCI) measurements of the user, identifying candidate brain activity-stimuli pairings from the BCI measurement having reliable correlation with predetermined stimuli, storing candidate brain activity-stimuli pairings, determining brain activity-stimuli pairings having reliable correlation when the user is imagining the stimuli, storing the brain activity-stimuli pairings having reliable correlation when the user is imagining the stimuli and retrieving and displaying the stimuli when a correlated BCI measurement is detected to perform telepathic computer control.

Example 34 may optionally include the subject matter of any one or more of Examples 14-33, wherein the presenting a set of stimuli to a user further includes presenting compound stimuli to the user to increase correlation reliability.

Example 35 may optionally include the subject matter of any one or more of Examples 14-34, wherein the telepathic computer control includes a telepathic search performed by the user by recreating mental imagery of a stimuli paired with a BCI measure associated with a search object.

Example 36 may optionally include the subject matter of any one or more of Examples 14-35, wherein the telepathic search is performed by matching the patterns of thought of a user to a database of content that is categorized to brain patterns of the user developed in response to brain activity measurements associated with the pattern of thought to produce search results and weighting the search results based on a number of elements in the patterns of thought that match with elements known to be associated with content in the database.

Example 37 may optionally include the subject matter of any one or more of Examples 14-36, wherein the telepathic search includes a search for an image, wherein the user thinks of the image that is an object of the search and providing results of images that matches brain activity-stimuli pairings to the user's thoughts of the image.

Example 38 may optionally include the subject matter of any one or more of Examples 14-37, wherein the telepathic search includes a search for a work of music, wherein the user thinks of sounds associated with the work of music and providing results of music that matches brain activity-stimuli pairings to the user's thoughts of the sounds associated with the work of music.

Example 39 may optionally include the subject matter of any one or more of Examples 14-38, wherein the telepathic search includes a telepathic search performed using a combination of multi-voxel pattern analysis (MVPA) and functional near infrared spectroscopy (fNIRS) to identify patterns of distributed brain activity correlated with a particular thought.

Example 40 may optionally include the subject matter of any one or more of Examples 14-39, wherein the telepathic computer control includes a telepathic communication, wherein at least two users trained with a common mental vocabulary use the common mental vocabulary to communicate with each other based on brain activity-stimuli pairings.

Example 41 may optionally include the subject matter of any one or more of Examples 14-40, wherein a sending user is identified on a user interface of a receiving user, and wherein a sending user thinks of a receiving user to select a receiving user to send a message.

Example 42 may optionally include the subject matter of any one or more of Examples 14-41, wherein the telepathic computer control includes a telepathic augmented reality (AR) performed by thinking about a brain activity-stimuli pairing that associates mental imagery with a model to perform a predetermined action.

Example 43 may optionally include the subject matter of any one or more of Examples 14-42, wherein the predetermined action includes presenting the user with sensory signals produced by an AR object associated with the brain activity-stimuli pairing, wherein the sensory signals includes visual, audio and tactile signals.

Example 44 may optionally include the subject matter of any one or more of Examples 14-43, wherein the predetermined action includes presenting an AR experience not purposefully invoked by the user through monitoring of BCI inputs.

Example 45 may optionally include the subject matter of any one or more of Examples 14-44, wherein the predetermined action includes directing movement of AR characters by thinking about a brain activity-stimuli pairing.

Example 46 may optionally include the subject matter of any one or more of Examples 14-45, wherein the predetermined action includes an action initiated using the brain activity-stimuli pairing with monitored environmental cues.

Example 47 may optionally include the subject matter of any one or more of Examples 14-46, wherein the performing a processor controlled function based on the user brain signatures includes operating computing devices by focusing, by the user, mental attention on different sections of a visual field of the user.

Example 48 may optionally include the subject matter of any one or more of Examples 14-47, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes dividing a mental desktop workspace into visuospatial regions based on regions of a field of view of a user, training a user to map a visual field of the user to regions of a primary visual cortex of the user, wherein the regions of the primary visual cortex correspond to one of the visuospatial regions, assigning content to physiologically segregated sections of the visual field represented by the visuospatial regions and accessing assigned information by mentally visualizing one of the visuospatial regions to access content assigned to the visualized visuospatial region.

Example 49 may optionally include the subject matter of any one or more of Examples 14-48, wherein the visuospatial regions includes a left and right hemifield for each of a left eye and a right eye, and wherein each hemifield is divided into an upper and lower division.

Example 50 may optionally include the subject matter of any one or more of Examples 14-49, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes imagining, by a user, movements of a body location associated with providing a computer input, recording brain activity emerging from a topographically organized brain region dedicated to controlling movements of the corresponding body location, correlating the recorded brain activity in the topographically organized brain region with the movement of the corresponding body location, performing a mental gesture by visualizing movement of the body location to produce activity in the topographically organized brain region, detecting brain activity corresponding to the recorded brain activity and performing a computer input associated with the movement of the corresponding body location in response to detection of the brain activity corresponding to the recorded brain activity.

Example 51 may optionally include the subject matter of any one or more of Examples 14-50, further including receiving perceptual computing input, wherein the performing a processor controlled function based on the user brain signatures includes correlating the temporal and spatial patterns of biophysical signals associated with brain activity with the perceptual computing input to determine an intent of the user and initiating a command to control electronic devices based on the determined user intent.

Example 52 may optionally include the subject matter of any one or more of Examples 14-51, wherein the receiving perceptual computing input includes receiving gesture, voice, eye tracking and facial expression input.

Example 53 may optionally include the subject matter of any one or more of Examples 14-52, wherein the receiving perceptual computing input comprises receiving at least one of: gesture, voice, eye tracking or facial expression input.

Example 54 may optionally include the subject matter of any one or more of Examples 14-53, wherein the correlating the gathered temporal and spatial patterns of biophysical signals further includes identifying a pattern to the temporal and spatial patterns of biophysical signals associated with brain activity from the user prior to initiating a command indicating that a next sensor-detected event is a command.

Example 55 may optionally include the subject matter of any one or more of Examples 14-54, further including receiving perceptual computing input, wherein the performing the processor controlled function further includes indicating a modality from the brain activity and perceptual computing inputs having precedence.

Example 56 may optionally include the subject matter of any one or more of Examples 14-55, further including measuring the temporal and spatial patterns of biophysical signals associated with brain activity of the user contemporaneously with receiving perceptual computing input, and using the contemporaneous temporal and spatial patterns of biophysical signals associated with brain activity of the user and the received perceptual computing input to reinforce an input command.

Example 57 may optionally include the subject matter of any one or more of Examples 14-56, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes measuring temporal and spatial patterns of biophysical signals associated with brain activity of the user, determining a state of the user based on the measured temporal and spatial patterns of biophysical signals associated with brain activity of the user and providing a response to the user based on the determined state.

Example 58 may optionally include the subject matter of any one or more of Examples 14-57, further including receiving perceptual computing input, wherein the perceptual computing input includes eye tracking to select a target, and wherein the temporal and spatial patterns of biophysical signals of the user are used to act on the target.

Example 59 may optionally include the subject matter of any one or more of Examples 14-58, wherein the performing a processor controlled function based on the user brain signatures further includes using temporal and spatial patterns of biophysical signals associated with brain activity of the user to interrupt a system responding to another modality.

Example 60 may optionally include the subject matter of any one or more of Examples 14-59, wherein the performing a processor controlled function based on the user brain signatures further includes using temporal and spatial patterns of biophysical signals associated with brain activity of the user to provide feedback to the user when the temporal and spatial patterns of biophysical signals associated with brain activity of the user have been identified and received.

Example 61 may optionally include the subject matter of any one or more of Examples 14-60, wherein the performing a processor controlled function based on the user brain signatures further includes alerting a system to change states when a change in state of a user is determined to have changed based on the correlating the gathered temporal and spatial patterns of biophysical signals.

Example 62 may optionally include the subject matter of any one or more of Examples 14-61, further including mapping the brain activity of the user to activation of a command window-of-opportunity when the brain activity occurs.

Example 63 may optionally include the subject matter of any one or more of Examples 14-62, further including obtaining perceptual computing inputs, gathering data from a database arranged to maintain heuristics on how perceptual computing inputs and the temporal and spatial patterns of biophysical signals of the user interrelate, analyzing the temporal and spatial patterns of biophysical signals of the user, the perceptual computing inputs, and the input from the database to determine user intent and generating a command based on the determined user intent.

Example 64 may optionally include the subject matter of any one or more of Examples 14-63, further including measuring environmental and user factors, determining possible interference, and adjusting temporal and spatial patterns of biophysical signals of the user based on the determined possible interference.

Example 65 may optionally include the subject matter of any one or more of Examples 14-64, wherein the processor controlled function comprises one selected from a group of actions consisting of performing a telepathic augmented reality (AR) by thinking about a brain activity-stimuli pairing that associates mental imagery with a model to perform a predetermined action, presenting an AR experience not purposefully invoked by the user through monitoring of BCI inputs, directing movement of AR characters by thinking about a brain activity-stimuli pairing, and an action initiated using the brain activity-stimuli pairing with monitored environmental cues.

Example 66 may include or may optionally be combined with the subject matter of any one of Examples 1-65 to include subject matter (such as means for performing acts or machine readable medium including instructions that, when executed by the machine, cause the machine to perform acts) including providing stimuli to a user, gathering temporal and spatial patterns of biophysical signals associated with brain activity in response to providing the stimuli to the user, correlating the gathered temporal and spatial patterns of biophysical signals associated with brain activity to identify user brain signatures and performing a processor controlled function based on the user brain signatures identified through correlating the gathered temporal and spatial patterns of biophysical signals associated with brain activity.

Example 67 may optionally include the subject matter of Example 66, wherein the providing stimuli to a user, gathering temporal and spatial patterns of biophysical signals associated with brain activity in response to providing the stimuli to the user and correlating the gathered temporal and spatial patterns of biophysical signals further includes calibrating a brain signature of a user based on the stimuli and authenticating the user by comparing a currently measured brain signature and the calibrated brain signature.

Example 68 may optionally include the subject matter of any one or more of Examples 66-67, wherein the calibrating the brain signature of the user includes presenting a set of stimuli to a user to incite brain activity responses, measuring brain anatomy and activity in response to the presented set of stimuli, performing pattern recognition of the measurements of brain anatomy and activity to produce a brain signature of the user, storing the produced brain signature and adding the stored brain signature to a database of anatomical and physiologic brain signatures of a predetermined population.

Example 69 may optionally include the subject matter of any one or more of Examples 66-68, wherein the authenticating the user includes presenting a previously applied set of stimuli to a user to incite brain activity responses, measuring brain anatomy and activity of the user based on the previously applied set of stimuli, performing pattern recognition of the measurements of brain anatomy and activity to produce a brain signature of the user and analyzing the brain signature of the user obtained through the performing the pattern recognition by comparing the brain signature with the stored brain signature of the user.

Example 70 may optionally include the subject matter of any one or more of Examples 66-69, wherein the performing a processor controlled function based on the user brain signatures includes directing a device to perform a function in response to the gathered temporal and spatial patterns of biophysical signals associated with the brain activity.

Example 71 may optionally include the subject matter of any one or more of Examples 66-70, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes presenting a set of stimuli to a user, obtaining brain-computer interface (BCI) measurements of the user, identifying candidate brain activity-stimuli pairings from the brain activity measurement having reliable correlation with predetermined stimuli, storing candidate brain activity-stimuli pairings, determining brain activity-stimuli pairings having reliable correlation when the user is imagining the stimuli, storing the brain activity-stimuli pairings having reliable correlation when the user is imagining the stimuli and retrieving and displaying the stimuli when a correlated brain activity measurement is detected to perform telepathic computer control.

Example 72 may optionally include the subject matter of any one or more of Examples 66-71, wherein the telepathic computer control includes a telepathic search performed by the user by recreating mental imagery of a stimuli paired with a BCI measure associated with a search object.

Example 73 may optionally include the subject matter of any one or more of Examples 66-72, wherein the telepathic search is performed by matching the patterns of thought of a user to a database of content that is categorized to brain patterns of the user developed in response to brain activity measurements associated with the patterns of thought to produce search results and weighting the search results based on a number of elements in the patterns of thought that match with elements known to be associated with content in the database.

Example 74 may optionally include the subject matter of any one or more of Examples 66-73, wherein the telepathic computer control includes a telepathic communication, wherein at least two users trained with a common mental vocabulary use the common mental vocabulary to communicate with each other based on brain activity-stimuli pairings.

Example 75 may optionally include the subject matter of any one or more of Examples 66-74, wherein a sending user is identified on a user interface of a receiving user.

Example 76 may optionally include the subject matter of any one or more of Examples 66-75, wherein a sending user thinks of a receiving user to select a receiving user to send a message.

Example 77 may optionally include the subject matter of any one or more of Examples 66-76, wherein the telepathic computer control includes a telepathic augmented reality (AR) performed by thinking about a brain activity-stimuli pairing that associates mental imagery with a model to perform a predetermined action.

Example 78 may optionally include the subject matter of any one or more of Examples 66-77, wherein the performing a processor controlled function based on the user brain signatures includes operating computing devices by focusing, by the user, mental attention on different sections of a visual field of the user.

Example 79 may optionally include the subject matter of any one or more of Examples 66-78, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes dividing a mental desktop workspace into visuospatial regions based on regions of a field of view of a user, training a user to map a visual field of the user to regions of a primary visual cortex of the user, wherein the regions of the primary visual cortex correspond to one of the visuospatial regions, assigning content to physiologically segregated sections of the visual field represented by the visuospatial regions and accessing assigned information by mentally visualizing one of the visuospatial regions to access content assigned to the visualized visuospatial region.

Example 80 may optionally include the subject matter of any one or more of Examples 66-79 further including receiving perceptual computing input, wherein the performing a processor controlled function based on the user brain signatures includes correlating the temporal and spatial patterns of biophysical signals associated with brain activity with the perceptual computing input to determine an intent of the user and initiating a command to control electronic devices based on the determined user intent.

Example 81 may optionally include the subject matter of any one or more of Examples 66-80, wherein the receiving perceptual computing input includes receiving gesture, voice, eye tracking and facial expression input.

Example 82 may optionally include the subject matter of any one or more of Examples 66-81, wherein the receiving perceptual computing input comprises receiving at least one of: gesture, voice, eye tracking or facial expression input.

Example 83 may optionally include the subject matter of any one or more of Examples 66-82, wherein the correlating the gathered temporal and spatial patterns of biophysical signals further includes identifying a pattern to the temporal and spatial patterns of biophysical signals associated with brain activity from the user prior to initiating a command indicating that a next sensor-detected event is a command.

Example 84 may optionally include the subject matter of any one or more of Examples 66-83, further including receiving perceptual computing input, wherein the performing the processor controlled function further includes indicating a modality from the brain activity and perceptual computing inputs having precedence.

Example 85 may optionally include the subject matter of any one or more of Examples 66-84, further including measuring the temporal and spatial patterns of biophysical signals associated with brain activity of the user contemporaneously with receiving perceptual computing input, and using the contemporaneous temporal and spatial patterns of biophysical signals associated with brain activity of the user and the received perceptual computing input to reinforce an input command.

Example 86 may optionally include the subject matter of any one or more of Examples 66-85, wherein the providing stimuli to a user, the gathering temporal and spatial patterns of biophysical signals associated with brain activity in response to providing the stimuli to the user, the correlating the gathered temporal and spatial patterns of biophysical signals and the performing a processor controlled function based on the user brain signatures further includes measuring temporal and spatial patterns of biophysical signals associated with brain activity of the user, determining a state of the user based on the measured temporal and spatial patterns of biophysical signals associated with brain activity of the user and providing a response to the user based on the determined state.

Example 87 may optionally include the subject matter of any one or more of Examples 66-86, further including receiving perceptual computing input, wherein the perceptual computing input includes eye tracking to select a target, and wherein the temporal and spatial patterns of biophysical signals of the user are used to act on the target.

Example 88 may optionally include the subject matter of any one or more of Examples 66-87, wherein the performing a processor controlled function based on the user brain signatures further includes using temporal and spatial patterns of biophysical signals associated with brain activity of the user to interrupt a system responding to another modality.

Example 89 may optionally include the subject matter of any one or more of Examples 66-88, wherein the telepathic computer control comprises one selected from a group of controls consisting of a telepathic search performed by the user by recreating mental imagery of stimuli paired with a BCI measure associated with a search object; a telepathic communication, wherein at least two users trained with a common mental vocabulary use the common mental vocabulary to communicate with each other based on brain activity-stimuli pairings; and a telepathic augmented reality (AR) performed by thinking about a brain activity-stimuli pairing that associates mental imagery with a model to perform a predetermined action.

Example 90 may optionally include the subject matter of any one or more of Examples 66-89, wherein the performing a processor controlled function based on the user brain signatures comprises one selected from a group of functions consisting of operating computing devices by focusing detected mental attention on different sections of a visual field of the user; providing a mental desktop by dividing a mental desktop workspace into visuospatial regions based on regions of a field of view of a user, training a user to map a visual field of the user to regions of a primary visual cortex of the user, wherein the regions of the primary visual cortex correspond to one of the visuospatial regions, assigning content to physiologically segregated sections of the visual field represented by the visuospatial regions and accessing assigned information by mentally visualizing one of the visuospatial regions to access content assigned to the visualized visuospatial region; correlating the temporal and spatial patterns of biophysical signals associated with brain activity with the perceptual computing input to determine an intent of the user and initiating a command to control electronic devices based on the determined user intent; performing a processor controlled function based on the user brain signatures by measuring temporal and spatial patterns of biophysical signals associated with brain activity of the user, determining a state of the user based on the measured temporal and spatial patterns of biophysical signals associated with brain activity of the user and providing a response to the user based on the determined state; and using temporal and spatial patterns of biophysical signals associated with brain activity of the user to interrupt a system responding to another modality.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplate are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure, for example, to comply with 37 C.F.R. §1.72(b) in the United States of America. It is submitted with the understanding that it will not be used to interpret or limit the scope or meaning of the claims. Also, in the above Detailed Description, various features may be grouped together to streamline the disclosure. However, the claims may not set forth features disclosed herein because embodiments may include a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

1. A system for providing a brain computer interface, comprising:
a library of stimuli for provisioning to a user;
a data collection device for gathering temporal and spatial patterns of biophysical signals associated with brain activity in response to provisioning stimuli from the library of stimuli to the user; and
a processing device for correlating the gathered temporal and spatial patterns of biophysical signals associated with brain activity to identify a brain signature of the user and performing a processor controlled function based on the brain signature of the user identified through correlating the gathered temporal and spatial patterns of biophysical signals associated with brain activity.

2. The system of claim 1, wherein the processing device builds a mental profile of the user as a function of the stimuli based on the temporal and spatial patterns of biophysical signals associated with brain activity of the user.

3. The system of claim 1, wherein the processing device provides identification and authentication of a user, wherein a mental profile of a user is created by the processing device during a calibrating stage based on presentation of stimuli from the library of stimuli to the user, the processing device further determining whether a mental profile of a user being authenticated correlates to the mental profile of the user created during the calibration stage.

4. The system of claim 1, wherein the processing device is arranged to perform telepathic contextual search by monitoring transmissions from a brain-computer interface system of a user, displaying stimuli associated with brain activity measurements from the user, searching for the brain activity measurements to locate a search object associated with the brain activity measurements and returning search results based on a match between the brain activity measurements and search objects having the associated brain activity measurements correlated therewith.

5. The system of claim 1, wherein the processing device provides telepathic augmented reality by receiving input from brain-computer interface sensors and detectors and a biometric and environmental sensor array, the processing device arranged to map input and data obtained a database of recognized sensor inputs, augmented reality character and augmented reality environmental content to an augmented reality experience, the processing device blending augmented reality characters with the environment and presenting the augmented reality experience to a user based on user intent derived from the input from the brain-computer interface sensors and detectors and the biometric and environmental sensor array.

6. The system of claim 1, wherein the processing device creates a mental desktop representing a left and right hemifield for each of a left and right eyes of a user, the processing device further segregating each eye into an upper division and a lower division, wherein the mental desktop comprises eight areas of a visual field of the user having information assigned thereto, the processing device detecting mentally visualization of a region in the mental desktop and implementing a function according to the information assigned to the mentally visualized region.

7. The system of claim 1, wherein the processing device is arranged to determine an intent of the user based on perceptual computing inputs, temporal and spatial patterns of biophysical signals associated with brain activity of the user and interrelatedness of the perceptual computing inputs and the temporal and spatial patterns of biophysical signals associated with brain activity of the user, wherein the processing device initiates a command based on the determined user intent.

8. A method for providing a brain computer interface, comprising:
providing stimuli to a user;
gathering temporal and spatial patterns of biophysical signals associated with brain activity in response to providing the stimuli to the user;
correlating the gathered temporal and spatial patterns of biophysical signals associated with brain activity to identify user brain signatures; and
performing a processor controlled function based on the user brain signatures identified through correlating of the gathered temporal and spatial patterns of biophysical signals associated with brain activity.

9. The method of claim 8, wherein the processor controlled function comprises determining at least one similarity between identified patterns of the user and patterns common to a group of users.

10. The method of claim 8, wherein the providing stimuli to a user, gathering temporal and spatial patterns of biophysical signals associated with brain activity in response to providing the stimuli to the user and correlating of the gathered temporal and spatial patterns of biophysical signals further comprises calibrating a brain signature of a user based on the stimuli and authenticating the user by comparing a currently measured brain signature and the calibrated brain signature.

11. The method of claim 10, wherein the authenticating the user comprises:
presenting a previously applied set of stimuli to a user to incite brain activity responses;
measuring brain anatomy and activity of the user based on the previously applied set of stimuli;
performing pattern recognition of the measurements of brain anatomy and activity to produce a brain signature of the user; and
analyzing the brain signature of the user obtained through the performing the pattern recognition by comparing the brain signature with the calibrated brain signature of the user.

12. The method of claim 11, wherein the analyzing the brain signature of the user includes comparing the brain signature with anatomical and physiologic brain signatures of a predetermined population.

13. The method of claim 8, wherein the processor controlled function comprises one selected from a group of functions consisting of:
a telepathic search performed by the user by recreating mental imagery of a stimuli paired with a brain computer interface measure associated with a search object;
a telepathic communication, wherein at least two users trained with a common mental vocabulary use the common mental vocabulary to communicate with each other based on brain activity-stimuli pairings; and
operating computing devices by focusing detected mental attention on different sections of a visual field of the user.

14. The method of claim 13, wherein the telepathic search is performed by matching the patterns of thought of a user to a database of content that is categorized to brain patterns of the user developed in response to brain activity measurements associated with the pattern of thought to produce search results and weighting the search results based on a number of elements in the patterns of thought that match with elements known to be associated with content in the database.

15. (canceled)

16. The method of claim 8, wherein the processor controlled function comprises one selected from a group of actions consisting of performing a telepathic augmented reality by thinking about a brain activity-stimuli pairing that associates mental imagery with a model to perform a predetermined action, presenting an augmented reality experience not purposefully invoked by the user through monitoring of brain computer interface inputs, directing movement of augmented reality characters by thinking about a brain activity-stimuli pairing, and an action initiated using the brain activity-stimuli pairing with monitored environmental cues.

17. (canceled)

18. The method of claim 8, wherein the providing stimuli to a user, the gathering temporal and spatial patterns of biophysical signals associated with brain activity in response to providing the stimuli to the user, the correlating of the gathered temporal and spatial patterns of biophysical signals and the performing a processor controlled function based on the user brain signatures further comprises accessing information using a mental desktop or using a mental gesture to provide computer input;
wherein the accessing information using a mental desktop comprises dividing a mental desktop workspace into visuospatial regions based on regions of a field of view of a user, training a user to map a visual field of the user to regions of a primary visual cortex of the user corresponding to one of the visuospatial regions, assigning content to physiologically segregated sections of the visual field represented by the visuospatial regions and accessing assigned information by mentally visualizing one of the visuospatial regions to access content assigned to the visualized visuospatial region; and
wherein the using a mental gesture to provide computer input comprises imagining, by a user, movements of a body location associated with providing a computer input, recording brain activity emerging from a topographically organized brain region dedicated to controlling movements of the corresponding body location, correlating the recorded brain activity in the topographically organized brain region with the movement of the corresponding body location, performing a mental gesture by visualizing movement of the body location to produce activity in the topographically organized brain region, detecting brain activity corresponding to the recorded brain activity and performing a computer input associated with the movement of the corresponding body location in response to detection of the brain activity corresponding to the recorded brain activity.

19-30. (canceled)

31. At least one machine readable memory comprising instructions that, when executed by the machine, cause the machine to perform operations for providing a brain computer interface (BCI) system based on gathered temporal and spatial patterns of biophysical signals, the operations comprising:
providing stimuli to a user;
gathering temporal and spatial patterns of biophysical signals associated with brain activity in response to providing the stimuli to the user;
correlating the gathered temporal and spatial patterns of biophysical signals associated with brain activity to identify user brain signatures; and
performing a processor controlled function based on the user brain signatures identified through correlating of the gathered temporal and spatial patterns of biophysical signals associated with brain activity.

32. The at least one machine readable memory of claim 31, wherein the performing a processor controlled function comprises determining at least one similarity between identified patterns of the user and patterns common to a group of users.

33. The at least one machine readable memory of claim 31, wherein the providing stimuli to a user, gathering temporal and spatial patterns of biophysical signals associated with brain activity in response to providing the stimuli to the user and correlating of the gathered temporal and spatial patterns of biophysical signals further comprises calibrating a brain signature of a user based on the stimuli and authenticating the user by comparing a currently measured brain signature and the calibrated brain signature.

34. The at least one machine readable memory of claim 33, wherein the authenticating the user comprises:
presenting a previously applied set of stimuli to a user to incite brain activity responses;
measuring brain anatomy and activity of the user based on the previously applied set of stimuli;
performing pattern recognition of the measurements of brain anatomy and activity to produce a brain signature of the user; and
analyzing the brain signature of the user obtained through the performing the pattern recognition by comparing the brain signature with the calibrated brain signature of the user.

35. The at least one machine readable memory of claim 34, wherein the analyzing the brain signature of the user includes comparing the brain signature with anatomical and physiologic brain signatures of a predetermined population.

36. The at least one machine readable memory of claim 31, wherein the performing a processor controlled function comprises one selected from a group of controls consisting of:
a telepathic search performed by the user by recreating mental imagery of stimuli paired with a brain computer interface measure associated with a search object,
a telepathic communication, wherein at least two users trained with a common mental vocabulary use the common mental vocabulary to communicate with each other based on brain activity-stimuli pairings; and
a telepathic augmented reality performed by thinking about a brain activity-stimuli pairing that associates mental imagery with a model to perform a predetermined action.

37. The at least one machine readable memory of claim 36, wherein the telepathic search is performed by matching the patterns of thought of a user to a database of content that is categorized to brain patterns of the user developed in response to brain activity measurements associated with the patterns of thought to produce search results and weighting the search results based on a number of elements in the patterns of thought that match with elements known to be associated with content in the database.

38. The at least one machine readable memory of claim 31, wherein the performing a processor controlled function based on the user brain signatures comprises one selected from:
correlating the temporal and spatial patterns of biophysical signals associated with brain activity with perceptual computing input to determine an intent of the user and initiating a command to control electronic devices based on the determined user intent;
performing a processor controlled function based on the user brain signatures by measuring temporal and spatial patterns of biophysical signals associated with brain activity of the user, determining a state of the user based on the measured temporal and spatial patterns of biophysical signals associated with brain activity of the user and providing a response to the user based on the determined state;
using temporal and spatial patterns of biophysical signals associated with brain activity of the user to interrupt a system responding to another modality;
operating computing devices by focusing detected mental attention on different sections of a visual field of the user; and
providing a mental desktop by dividing a mental desktop workspace into visuospatial regions based on regions of a field of view of a user, training a user to map a visual field of the user to regions of a primary visual cortex of the user, wherein the regions of the primary visual cortex correspond to one of the visuospatial regions, assigning content to physiologically segregated sections of the visual field represented by the visuospatial regions and accessing assigned information by mentally visualizing one of the visuospatial regions to access content assigned to the visualized visuospatial region.

39. The method of claim 8 further comprising measuring environmental and user factors, determining possible interference, and adjusting temporal and spatial patterns of biophysical signals of the user based on the determined possible interference.
